(19) United States
(12) Patent Application Publication
(10) Pub. No.: US 2024/0415156 A1
(43) Pub. Date: Dec. 19, 2024

(54) EMULSIONS, COMPOSITIONS FOR EMULSIONS, METHODS FOR MAKING THE SAME AND USES THEREOF

(71) Applicant: ADIPOSS SA, Plan-les-Ouates (CH)

(72) Inventors: Nathalie STRANSKY-HEILKRON, Plan-les-Ouates (CH); Andrej BABIC, Plan-les-Ouates (CH)

(21) Appl. No.: 18/704,879

(22) PCT Filed: Oct. 28, 2022
(86) PCT No.: PCT/EP2022/080246

§ 371 Date: Apr. 25, 2024

(30) Foreign Application Priority Data
Oct. 28, 2021 (EP) 21205260.9

(51) Int. Cl. A23L 29/10; A23L 2/52; B01F 23/41; B01F 33/501; B01F 101/14

(52) U.S. Cl. CPC A23L 29/10; A23L 2/52; B01F 23/4105; B01F 23/4143; B01F 23/4145; B01F 33/50111; B01F 2101/14

(57) ABSTRACT

The present invention provides emulsions as well as compositions and methods for making the same, which are characterized by an ester component, a non-ionic surfactant component and a water component and wherein emulsification using a low or ultra-low energy method such as shaking by hand is possible to obtain a storage stable emulsion with small particle sizes.

### FIELD OF THE INVENTION

The invention provides emulsions and especially stable nanoemulsions that can be obtained with a surfactant, no solvent or co-solvent and an ultra-low energy method of preparation such as shaking by hand, uses thereof, a water-containing composition and a water-free premix for preparing the emulsions of the invention and a production method thereof.

### BACKGROUND OF THE INVENTION

Emulsions are defined as dispersions of two immiscible liquids, with the droplets forming the dispersed phase, whereas the liquid surrounding them forms the continuous phase¹. The commonly used liquids to form emulsions are purified water and oil. The oil droplets dispersed in an aqueous phase are known as oil-in-water (o/w) emulsions. These emulsion systems can be used for the delivery of hydrophobic active substances. The water droplets dispersed in oil are called the water-in-oil (w/o) emulsions and are used for the delivery of hydrophilic compounds.

Emulsions are categorized as coarse emulsions, microemulsions and nanoemulsions based on their droplet size and stability. The coarse emulsions are also known as conventional emulsions or macroemulsions. They have particle size of diameter >200 nm range and are thermodynamically metastable. They break down over time due to various destabilizing factors. Conventional emulsions are optically turbid as the droplet size is similar to that of the wavelength of light and hence, the droplets scatter the incident light and the emulsion appears opaque. The droplets in microemulsions are <100 nm in size. Microemulsions are thermodynamically stable. However, their stability may be affected by even slight variations in environmental conditions such as composition, temperature and pH. Microemulsions form spontaneously. Since the particle size is lower than the wavelength of light, they are optically transparent or translucent. Nanoemulsions have droplet dimensions similar to those of microemulsions, typically being <300 nm and in some cases <200 nm or even <100 nm or less. Similar to conventional emulsions, nanoemulsions are thermodynamically metastable as phase separation occurs over time. However, nanoemulsions are conferred with kinetic stability as there is no gravitational separation and droplet aggregation due to the reduced attractive force between the small sized droplets. Nanoemulsions, unlike the thermodynamically stable microemulsions, are typically not affected by physical and chemical variations including temperature and pH.

Nanoemulsion formulation requires the use of two immiscible liquids and one or more emulsifiers. The oil phase is typically made of non-polar components, such as vegetal, mineral or animal oil or fats, or other non-polar solvents. Non-polar essential oils, lipid substitutes, resins, waxes, weighting agents, oil-soluble vitamins, and various lipophilic components can also be used as components of the oil phase. The aqueous phase of a nanoemulsion is usually made of a polar solvent and at least one co-solvent. The most commonly used polar solvent is purified water, with optional addition of salts to obtain a buffered aqueous solution (buffer). Carbohydrates, protein, alcohol, and polyols are used as co-solvents. Emulsions can breakdown due to Ostwald ripening (increase in mean droplet size over time), flocculation, coalescence, and gravitational separation. This can be prevented by adding a stabilizing agent. The stabilizers distribute on the particles and can form either a monolayer, multilayer, or solid particulate nanoemulsions. Some of the stabilizers used in the state-of-the-art nanoemulsions are surfactants, weighting agents, ripening retarders, and texture modifiers.

Surfactants are surface-active molecules. They reduce the interfacial tension resulting in the formation of small and stable nanoemulsions. Surfactants also prevent collision and coalescence between the droplets and increases the kinetic stability of the (nano)emulsions. They can be cationic, anionic, non-ionic, or zwitterionic in nature. Some examples of emulsifiers are small-molecule surfactants, phospholipids, proteins, and polysaccharides. Two or more surfactants can be used together in (nano)emulsion formulation for their synergistic effects.

Nanoemulsions have numerous droplets which increases the interfacial surface area of these systems. A large amount of energy is required to create such large interfacial surface areas. Thus, nanoemulsion formation is not spontaneous but requires energy input. Nanoemulsions can be prepared by either high- or low-energy methods. The high-energy methods involve the use of mechanical devices such as high-pressure valve homogenizers, microfluidizers and ultrasonicators that disrupt the oil phase to make it interact with the water phase and form smaller oil droplets.

Nanoemulsion formation by low-energy methods relies on physicochemical properties such as temperature, composition, and solubility². The phase inversion temperature method consists in mixing the oil, water and surfactant at room temperature and then gradually increasing the temperature of the mixture (addition of energy). Above the so-called phase inversion temperature, the surfactant solubility changes from the aqueous phase to the oil phase, resulting in a water-in-oil emulsion. The mixture is then quickly cooled down to obtain an oil-in-water nanoemulsion.

Spontaneous emulsification can take place when mixing two liquid phases at room temperature³. One of the phases is purely aqueous; the other one is a mixture of oil, surfactant and a water-miscible co-solvent. The solvent displaces itself from the oily to the aqueous phase to thereby induce great turbulence at the water/oil interface. The two liquids, thermodynamically stable alone, are brought to a non-equilibrium state when they are mixed. Thus, the rapid transfer of hydrophilic materials from the oil to the water phase results in a dramatic increase of the interfacial area, giving rise to the metastable emulsion state. In order to obtain nanometric-scaled droplets with this spontaneous emulsification method, the experimental conditions commonly reported in literature involve a very high solvent/oil ratio (e.g. a very low percentage of oil in the organic phase before mixture). The solvent diffusion is hence even quicker, and the turbulence thereby generated causes nano-scaled droplets to form.

Self-emulsifying drug delivery systems (SEDDS) have also been developed to administer molecules with low water solubility⁴. They are made of oil, surfactant and cosurfactants and/or cosolvents formulated in a tablet or soft- or hard-shell capsule. Following oral administration, an emulsion is formed in the gastrointestinal tract, leading to higher solubility, protection from enzymatic degradation and improved intestinal absorption. These systems are also termed self-micro-emulsifying or self-nano-emulsifying drug delivery systems (SMEDDS or SNEMDDS) when the lipidic particles formed after administration are below 100 nm or 200 nm, respectively. The oil phase is usually made of medium- or long-chain triglycerides. In addition to a surfactant, cosurfactants or cosolvents are added to the formulation of SEDDS. The most common ones are propylene glycol, ethanol, glycerol and polyethylene glycols (PEG).

An example of marketed self-microemulsifying drug delivery system is Sandimmune Neoral. It contains cyclosporine (active pharmaceutical ingredient), ethanol (solvent), glycerol monolinoleate (co-surfactant), propylene glycol (co-solvent), and polyoxyl 40 hydrogenated castor oil (surfactant), filled into soft capsules.

It is a common general feature of the state-of-the-art emulsions that they are characterized by a rather complex compositions with multiple components and/or that they require high energy manufacturing methods and/or that they are not stable over extended periods of time. There is, therefore, a need for providing simple emulsions that can be prepared with a low energy method and that are stable over extended periods of time.

### SUMMARY OF THE INVENTION

The invention has the objective of overcoming the problems and shortcomings of the state of the art. Specifically, it is an objective of the present invention to provide stable emulsions that can be prepared by a low-energy manufacturing process such as shaking by hand. It is a further objective to provide emulsions which do not require complex compositions with multiple stabilizers, multiple surfactants and co-surfactants, solvents, co-solvents or the like. Another objective is to provide emulsions that are characterized by a narrow distribution of particle sizes, i.e. a low polydispersity index. It is a further objective of the present invention to provide emulsions that are suitable as carriers for active agents in the fields of cosmetics and/or foodstuffs. Of course, it is also an objective to provide emulsions that accomplish two or more of the above-mentioned objectives. It is yet another objective of the present invention to provide means and methods for preparing the emulsions of the present invention.

Surprisingly, advantageous emulsions that are typically highly homogenous, low polydispersed emulsions with high oil phase content can be obtained by the very low energy process of shaking by hand at room temperature when using compositions containing a particular ester component. Such emulsions typically display particle size between 20 and 400 nm and a polydispersity index of 0.40 or less. Surprisingly, emulsions that remain stable (in appearance and particle size) at room temperature for several weeks or months can be obtained with a surfactant.

The present invention is based on these findings. It thus accomplishes the above objectives by the technical teachings specified hereinbelow and in the appended claims. Specifically, the invention provides an emulsion as defined in appended claim 1. The invention further provides a product for emulsification as specified in appended claim 2. The invention further provides a premix for preparing the emulsions of the invention. This premix is specified in the appended claim 3. In yet another aspect, the invention provides methods for making the emulsions of the present invention. Such methods are specified in the appended claim 4. Preferred aspects of the different embodiments of the invention are specified in the dependent claims 5 to 15. Further preferred aspects and ways of implementing the invention are provided in the detailed description below.

### DETAILED DESCRIPTION OF THE INVENTION

### BRIEF DESCRIPTION OF FIGURES

FIGS. 1, 2 and 3 show ternary diagrams for the emulsions of Examples 3, 4 and 5, respectively. The abbreviation “w” stands for the content of water component, ranging from 0 wt. % at the top of the diagram to 100 wt. % at the lower right corner. The abbreviation “s” stands for the content of surfactant component, ranging from 0 wt. % at the lower left corner of the diagram to 100 wt. % at the top. The abbreviation “o” stands for the content of ester component, ranging from 0 wt. % at the lower right corner of the diagram to 100 wt. % at the lower left corner. The grey areas within the diagram characterize compositional ratios for which the formation of a stable emulsion was observed. For these diagrams, stable emulsions were defined as stable for at least 24 hours.

### DEFINITIONS

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. According to one specific embodiment, the term “comprise” is meant to have the more limiting meaning of “consisting of”.

As used in the specification and claims, the singular forms “a”, “an” and “the” include plural references unless the context clearly dictates otherwise.

Descriptions of compounds herein are to be understood using generally accepted terminology for chemical names or structural formulae such as reflected, for instance, by the IUPAC “Gold Book” (status of Oct. 26, 2021) and related publications. Unless specified otherwise or the context dictates otherwise, compounds described herein are generally stable compounds without charges having with all atoms' valences being saturated by covalent bonds to neighboring atoms. According to standard practice in chemistry, structural formulae may be drawn by omitting hydrogen atoms that are bonded to carbon atoms. Free valences of carbon atoms are thus to be understood as being saturated by hydrogen atoms.

Unless specified otherwise or the context dictates otherwise, all method steps described herein may be carried out at any temperature at which the components involved show a suitable viscosity. This includes in particular the temperature range of 15 to 35° C., more preferably 18 to 30° C. and most preferably 20 to 25° C. In the context of the present invention, “room temperature” means a temperature of 22° C.

Unless specified otherwise or the context dictates otherwise, all relative amount indications are indicated on a weight-by-weight basis. Unless specified otherwise or the context dictates otherwise, the basis for the relative amount indication is the total weight of the composition, for which the relative amount indication is given.

The term “oil” as used herein in relation to emulsions refers to the ester component specified hereinbelow with reference to formulae (I), (A), (B), (C), (D) or (E) plus any further component that dissolves in the phase formed by the ester component.

The term “geminal”, as used herein, refers to the relationship between two atoms or functional groups that are attached to the same atom.

The related term “vicinal” refers to the relationship between two atoms or functional groups that are attached to adjacent atoms.

A statement “if multiple iodine atoms are present, it is required that the iodine atoms are neither geminal nor vicinal” as used herein means that there must not be any pair of iodine atoms that is geminal or vicinal, as defined above.

As used herein, the term “alkyl” includes any straight-chained or branched aliphatic saturated hydrocarbon group. Unless specified otherwise, alkyl groups may have any of from 1 to 20 carbon atoms, preferably 1 to 10 carbon atoms, more preferably 1 to 6 carbon atoms and most preferably 1 to 4 carbon atoms. Preferred alkyl groups are in particular methyl, ethyl, propyl, isopropyl, n-butyl, sec-butyl, and tert-butyl.

As used herein, the term “alkenyl” characterizes any straight-chained or branched aliphatic hydrocarbon group containing one or more carbon-carbon double bonds. Each carbon-carbon double bond may independently be in the E- or Z-configuration. Unless specified otherwise, alkenyl groups may have any of from 2 to 20 carbon atoms, preferably 2 to 10 carbon atoms, more preferably 2 to 6 carbon atoms and most preferably 2 to 4 carbon atoms.

Some of the compounds described herein may form different regioisomers and/or stereoisomers. Unless expressly specified or unless the context dictates otherwise, each reference to a compound forming different isomers should be understood as a reference to each of the possible isomers in pure form and also as a reference to a mixture of the possible isomers. For example, if the compound contains one chiral centre, the compound can be provided as a single isomer (R or S) or as a mixture of isomers, for example a racemic mixture. Where the compound contains more than one chiral centre, the compound can be provided as an enantiomerically pure diastereoisomer or as a mixture of two or more different enantiomers and/or diastereoisomers.

As used herein, the term “alkynyl” characterizes any straight-chained or branched aliphatic hydrocarbon group containing one or more carbon-carbon triple bonds. Unless specified otherwise, alkynyl groups may have any of from 2 to 20 carbon atoms, preferably 2 to 10 carbon atoms, more preferably 2 to 6 carbon atoms and most preferably 2 to 4 carbon atoms. The term “alkoxy” represents —O-alkyl. An example of an alkoxy is a C1-C6 alkoxy, which represents a straight or branched alkyl chain having from one to six carbon atoms attached to an oxygen atom. Exemplary C1-C6 alkoxy groups include methoxy, ethoxy, propoxy, isopropoxy, butoxy, sec-butoxy, t-butoxy, pentoxy, hexoxy, and the like. C1-C6 alkoxy includes within its definition a C1-C4 alkoxy group as a specific embodiment.

The term “aromatic group” as used herein refers to a carbocyclic or heterocyclic, aromatic, 5-14 membered monocyclic or polycyclic ring. It thus includes C₅₋₁₄ aryl groups as well as 5-14-membered heteroaryl groups. Heteroaryl groups may contain 1, 2, 3 or 4 heteroatoms independently selected from N, O and S. Exemplary aryls include phenyl, naphthyl, anthryl, phenanthryl. Exemplary heteroaryls include thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl. As used herein, the terms “aryl” and “heteroaryl” are intended to encompass also aromatic groups containing one or more atoms that do not participate in the aromatic system with delocalized electrons. Typical examples are a fluorenyl group or a 9,10-dihydrophenanthrenyl group.

In organic chemistry, a “saturated” compound is a chemical compound that has a chain of carbon atoms linked together by single bonds. Alkanes are saturated hydrocarbons. An “unsaturated” compound is a chemical compound that contains carbon-carbon double bonds or triple bonds, such as those found in alkenes or alkynes, respectively. Saturated and unsaturated compounds need not consist only of a carbon atom chain. They can form straight chain, branched chain, or ring arrangements. They can have functional groups, as well. It is in this sense that fatty acids are classified as saturated or unsaturated. The amount of unsaturation of a fatty acid can be determined by finding its iodine number.

Unsaturated compounds are those in which addition reaction can be obtained. In a chain of carbons, such as a fatty acid, a double or triple bond will cause a kink in the chain. These kinks have macro-structural implications. Unsaturated fats tend to be liquid at room temperature, rather than solid, as the kinks in the chain prevent the molecules from packing closely together to form a solid.

In the context of the present invention, the term “halogen” is used to refer to an atom selected from fluorine, chlorine, bromine and iodine.

In the context of the invention, the wording “moiety derived from” is meant to characterize a moiety that is identical to the compound from which it is derived with the sole exception that one or more hydrogen atoms of said compound are replaced by one or more covalent bonds for bonding to the adjacent moiety or moieties.

In the context of the present invention, the term “high energy method” typically refers to methods requiring the use of devices such as high-pressure homogenizers, microfluidizers and ultrasonicators that disrupt the oil phase to make it interact with the water phase and form smaller oil droplets. The term “low energy method” typically refers to methods that relies on physicochemical properties such as temperature, composition, and solubility², such as phase inversion temperature and self-emulsification.

In particular, the term “high energy method” refers to any method with an energy density of more than 10⁷ Pa (or J/m³).⁵ The term “low energy method” refers to any method with an energy density of less than 10⁶ Pa (or J/m³). The term “ultra-low energy method” characterizes methods that require particularly low amounts of energy density of less than 105 Pa (or J/m³); such as shaking by hand or methods with the same or similar input energy density. In addition, the emulsion prepared using an ultra-low energy method is typically obtained in 120 s or less, preferably 60 s or less, more preferably 30 s or less. Where a device is used to carry out an emulsification method, the energy density can be determined by converting the power consumption of the device into energy density. The information provided in section 2.5 of D. M. Lloyd et al., Journal of Food Engineering 166 (2015) 316-324 can be used for determining power consumption. Shaking by hand is always to be regarded as an ultra-low energy method.

Unless the context dictates otherwise, the terms “size”, “droplet size” or “particle size” refer to the Z-average diameter of droplets/particles of the dispersed phase in an emulsion. It is experimentally determined as the intensity weighted mean hydrodynamic size of the ensemble collection of particles measured by dynamic light scattering (ISO 22412:2017) using a Zetasizer Nano ZS (Malvern Panalytical Ltd, United Kingdom). The analyses are performed using a 4 mW He—Ne Laser (633 nm) at a 1730 scattering angle at 25° C. The size distribution and mean diameter are calculated from the data using ZetaSizer software v8. A more detailed description of the measurement method of dynamic light scattering is given in the examples section.

The term “polydispersity index” characterizes the width of a size distribution. In the context of the present invention, it is defined as the squared ratio between standard deviation and average value of the droplet size, i.e. PDI=s²/D² with s being the standard deviation and D being the average particle size. It is experimentally determined by dynamic light scattering (ISO 22412:2017) using the following experimental procedure and equipment:

PDI is measured using dynamic light scattering on an ISO 22412:2017 compliant Zetasizer Nano ZS (Malvern Panalytical Ltd, United Kingdom). The analyses are performed using a 4 mW He—Ne Laser (633 nm) at a 173° scattering angle at 25° C. The size distribution and mean diameter are calculated from the data using ZetaSizer software v8.

In the context of the present invention, a container is a rigid or semi-rigid receptacle, typically made of glass, plastic or metal or other suitable material, that can store a liquid or semi-solid product and allows the transport of such product contained within. It can present additional features such as a narrow neck, a closure device such as a stopper, cap or top, a spray nozzle, or a dropper. It can also feature dual or multiple compartments and/or chambers.

The term “polyhydroxy” or “polyhydric” refers to a chemical compound or moiety containing two or more hydroxyl groups per molecule.

An “emulsion” is a mixture of two or more liquids that are immiscible owing to phase separation, in which one phase is dispersed in small entities (e.g. droplets) in the other phase. The two immiscible liquids are generally termed the “water phase” and the “oil phase”. An oil and water emulsion can result in either an oil-in-water or a water-in-oil emulsion. In addition to the two immiscible liquids, emulsions usually require the addition of one or more stabilizing agents such as surfactants and co-solvent(s).

The term “between”, when used in connection with a numerical range, is intended to encompass also the specified limiting values. For instance, a content indication of between 5 wt. % to 60 wt. % is meant to encompass also a content of 5 wt. % and a content of 60 wt. % as embodiments of the invention.

Commercially available surfactants frequently consist of a mixture of individual surfactant compounds differing from each other for instance by virtue of the chain length of a hydrophilic group such as a polyoxyethylene group. When referring to a “single surfactant”, and unless the context dictates otherwise, the present application intends to refer either to a single surfactant compound or to a single surfactant commercial product, which may consist of a mixture of individual surfactant compounds, as indicated above.

Unless specified otherwise or the context dictates otherwise, the term “shaking” is intended to refer to any movement that includes at least one movement period of acceleration and one movement period of deceleration. References to a “frequency” of the shaking movement are to be understood as the number of complete cycles of the shaking movement per time unit. For example, in the case of a simple “up and down” shaking movement, a frequency indication characterizes a number of individual movements per time unit, in which the subject of the shaking procedure, such as a container filled with liquid, is moved from a starting position away to a first extreme position (e.g. upper turning point), further moved to a second extreme position (e.g. lower turning point) and then returned to its initial position. Analogous considerations apply for more complex shaking trajectories.

Unless specified otherwise or the context dictates otherwise, the term “water” is intended to refer to any grade or purity of water. Of course, the employed grade or purity of water must be compatible with the formation of emulsions and suitable for the envisaged use of the emulsion. For instance, if it is intended to use the emulsion for administration to humans, the water employed must be of a grade suitable for human consumption, i.e. food grade or pharmaceutical grade.

Unless specified otherwise or the context dictates otherwise, the term “purified water” refers to water that has a conductivity of less than 100 μS/cm, preferably 0.03 to 50 μS/cm and more preferably 0.05 to 10 μS/cm. By contrast, water that is not purified typically has a conductivity of more than 100 μS/cm and 2000 μS/cm or less, such as 200 μS/cm to 1000 μS/cm and more specifically 300 μS/cm to 800 μS/cm. Conductivity may be determined as described in the US Pharmacopoeia 26 (2003) 2141-2 (645).

### Overview

In a first embodiment, the present invention is based on the surprising discovery that high quality emulsions can be obtained by a low energy emulsification step and preferably an ultra-low energy emulsification step, such as shaking and typically shaking by hand.

Hence, in one aspect, the present invention provides a method for producing emulsions, which relies on said low energy emulsification. The method requires the use of a suitable composition of starting materials. It comprises three essential constituents: water, a surfactant and an ester component. Additional optional components are neither required nor excluded.

The emulsions obtained by the method of the present invention are characterized by various beneficial characteristics such as one or more of the following: they exhibit small particle sizes, they exhibit a narrow particle size distribution, and/or they exhibit favourable stability. The low and ultra-low energy emulsification is furthermore advantageous insofar as it does not require expensive or specialized equipment, so that it may be performed virtually anywhere including especially by patients and by end-consumers. A further benefit associated with method and composition is that there is no need for a large number of components.

The emulsions obtained by the method of this embodiment of the present invention can be used in various applications, including for instance, as a CT or PET-CT contrast agent, as a cosmetic or as a food supplement. Of course, depending on the intended use, an appropriate active agent must be present in the emulsion.

Another aspect of this embodiment of the invention relates to a composition for forming the emulsion of the first embodiment of the invention and a related ready-for-emulsification product. Said product contains said composition comprising all components of the final emulsion, but which is not yet in emulsified state. The product contains said composition in a closed container and it is provided with instructions for shaking the container by hand to thereby form an emulsion.

Yet another aspect of this embodiment of the present invention provides a premix for use as a starting material in the method of the invention. Said premix is a composition, which contains at least the surfactant and the ester component and optionally further components, but not the water component. Said premix may be provided in a container and it may contain instructions for addition of a water component, followed by shaking by hand. The emulsion of the invention may be prepared in a convenient manner by combining the premix with water or an alternative water-containing liquid such as juice, followed by the above-mentioned low-energy emulsification step such as shaking and especially shaking by hand. The water-free premix presents several advantages. The oil and surfactant can be mixed in advance, irrespective of the location of emulsion administration. The final users of the emulsion only have to add the water phase and shake the product a short time to obtain a homogeneous emulsion according to the invention, which typically is stable, has small particle sizes and low to very low polydispersity. As no water is present in the product provided to the final user, i.e. the premix, the shelf-life of the product is considerably extended without the use of preservatives to prevent microbial growth. In addition, storage and shipping of the product are facilitated thanks to the lower volume and weight of the product due to the absence of water.

The second embodiment of the present invention is based on the surprising discovery that a component employed in prior art emulsions as an active agent has a beneficial effect on structure and stability of emulsions and is thus suitable for use as an emulsion-forming agent even when used in modified form. More concretely, the iodine-containing CT contrast agent known from WO 2019/030024 A1 and WO 2020/165349 A1 can be used as a structural component in emulsions for carrying other active agents even when omitting the iodine atoms forming an essential element of the above-mentioned CT contrast agent of the prior art.

By consequence, in a first aspect of the second embodiment of the invention, an emulsion is provided, which contains an active agent (e.g. cosmetic agent) other than an iodine-containing compound like the CT contrast agent of the cited prior art.

According to a further aspect of the second embodiment, a method for producing said emulsion is provided. Said method may involve a low-energy emulsification step as described for the first embodiment. However, alternative ways of emulsification involving higher energy input are also encompassed.

Further aspects of the second embodiment provide a ready-for-emulsification product and a premix similar to those of the first embodiment. The second embodiment furthermore provides various uses of said products.

The third embodiment of the present invention is based on the surprising recognition that particularly advantageous emulsions can be obtained when using a composition of starting materials, wherein the other components are suitably selected. This involves for instance the use of particularly suitable surfactants, the omission of further surfactants and/or the use of beverages as water component.

In different aspects of this embodiment, emulsions, methods for their manufacture, a ready-for-emulsification product and a premix as well as various uses thereof are provided.

### DETAILED DESCRIPTION OF THE INVENTION

### Description of Components

### Ester Component

Depending on the embodiment and the degree of preference, different ester components may be employed. These are described in the following.

Definition E1: The ester component is characterized by the general Formula I.

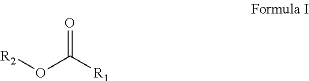

In general Formula I, R₁ represents a hydrocarbon group, which contains 30 or less carbon atoms, preferably 5-23 carbon atoms, more preferably 13-19 carbon atoms, and particularly preferably 15-17 carbon atoms. R₁ is typically selected from the group consisting of linear-chain alkyl groups, branched-chain alkyl groups, linear-chain alkenyl groups, branched-chain alkenyl groups, linear-chain alkynyl groups, branched-chain alkynyl groups. Each of these groups may contain one cyclopropyl group or cyclobutyl group. This small cyclic group may be incorporated at any position, so that any of the following arrangements is possible: C₁-Q₁, Q₁-C₁ and Q₁-C₁-Q₁, wherein C₁ represents the cyclopropyl or cyclobutyl group and each Q₁ represents a group independently selected from a linear-chain alkyl group, branched-chain alkyl group, linear-chain alkenyl group, branched-chain alkenyl group, linear-chain alkynyl group or branched-chain alkynyl group, wherein the above indications on carbon atom numbers apply to the entire group including cyclic and linear and/or branched parts. R₁ is preferably selected from the group consisting of linear-chain alkyl groups, branched-chain alkyl groups, linear-chain alkenyl groups, branched-chain alkenyl groups. The alkyl, alkenyl or alkynyl groups are optionally substituted by 1 to 6 substituents, preferably by 1 to 4 substituents, most preferably 1 to 3 substituents. Suitable substituents are selected from C₁₋₄-alkoxy, C₁₋₄-thioether, CN, N₃, halogen, such as fluorine, chlorine, bromine and iodine, preferably from fluorine, chlorine and iodine, and most preferably iodine. Hence, preferred ester compounds of formula I contain a group R₁ that is unsubstituted or is substituted by one, two, three, four, five or six iodine and/or fluorine, chlorine, bromine atoms as substituent. If multiple iodine atoms are present, the iodine atoms should neither be in geminal nor vicinal positions with respect to each other.

R₂ represents a group selected from the group consisting of linear-chain C₁₋₁₂ alkyl groups, branched-chain C₃₋₁₂ alkyl groups, cyclic C₃₋₁₂ alkyl groups, linear-chain C₂₋₁₂ alkenyl groups, branched-chain C₃₋₁₂ alkenyl groups, cyclic C₅₋₁₂ alkenyl groups, linear-chain C₂₋₁₂ alkynyl groups, branched-chain C₄₋₁₂ alkynyl groups, C₅₋₁₄ aryl groups, 5-14-membered heteroaryl groups and 5-14 membered saturated or partly unsaturated non-aromatic heterocycles with 1 to 4 heteroatoms independently selected from N, O and S. R₂ preferably is selected from the group consisting of linear-chain C₁₋₁₂ alkyl groups and branched-chain C₃₋₁₂ alkyl groups, and more preferably linear-chain C₁₋₁₂ alkyl groups. More preferably, R₂ is a linear C₁₋₄ alkyl group, a branched C₃₋₄ alkyl group, a linear C₂₋₄ alkenyl group, a branched C₃₋₄ alkenyl group, a linear C₂₋₄ alkynyl group or a branched C₄ alkynyl group. According to particularly preferred embodiments, R₂ is a linear-chain alkyl group that has 1-4 carbon atoms or a branched-chain alkyl group that has 3-4 carbon atoms. Any of these alkyl, alkenyl, alkynyl, aryl, heteroaryl or non-aromatic heterocyclic groups may be optionally substituted, i.e. it may optionally comprise 1 to 4 substituents as specified below. Preferably, the R₂ group is not substituted. Most preferably, R₂ is an unsubstituted group selected from a linear C₁₋₄ alkyl group, a branched C₃₋₄ alkyl group, a linear C₂₋₄ alkenyl group, a branched C₃₋₄ alkenyl group, a linear C₂₋₄ alkynyl group and a branched C₄ alkynyl group.

Suitable substituents of any of the above-mentioned R₂ groups may be selected from the group consisting of halogen, linear C₁₋₆ alkyl, branched C₃₋₆ alkyl, cyclic C₃₋₆ alkyl, linear C₂₋₆ alkenyl, branched C₃₋₆ alkenyl, cyclic C₅₋₆ alkenyl, linear C₂₋₆ alkynyl, branched C₄₋₆ alkynyl, C₅₋₁₄ aryl, 5-14-membered heteroaryl, cyano, thiol, thioether, nitro, azido, and hydroxyl, with the proviso that if each of the substituent and the substituted group is a saturated or unsaturated aliphatic group (which may include a saturated or unsaturated cyclic group but not a cyclic aromatic group), instead of the individual carbon atom restrictions, the total number of carbon atoms of the substituted group must be in accordance with the carbon atom range resulting from an addition of the permitted carbon atom numbers for the substituent and the substituted group. For example, if an alkyl group carries an alkyl substituent, the total number of carbons may be in the range of (1+1) to (12+6), i.e. from 2 to 18 carbons are permitted. The same applies to combinations of alkyl and alkenyl groups, alkyl and alkynyl groups, alkenyl and alkynyl groups and so forth. For other combinations of substituent group and substituent, in which it is clear for each atom whether it belongs to substituent or substituted group, the above carbon atom restrictions apply individually to the respective group. For instance, if an alkyl group is substituted by an aryl group, the alkyl group must have from 1 to 12 carbon atoms (and preferably 1 to 4 carbon atoms), whereas the aryl group must have 5-14 carbon atoms.

Further suitable substituents are represented by one of the formulae -M-L-R₄, -L-M-R4, -L-M-L-R₄ and -M-L-M-R₄. In one aspect, R₄ is selected from the group consisting of, linear C₁₋₁₂ alkyl, branched C₃₋₁₂ alkyl, cyclic C₃₋₁₂ alkyl, linear C₂₋₁₂ alkenyl, branched C₃₋₁₂ alkenyl, cyclic C₅₋₁₂ alkenyl, linear C₂₋₁₂ alkynyl, branched C₄₋₁₂ alkynyl, C₅₋₁₄ aryl, 5-14-membered heteroaryl, hydrogen and 5-14 membered saturated or partly unsaturated non-aromatic heterocycles with 1 to 4 heteroatoms independently selected from N, O and S, or combinations thereof provided that the total number of carbon atoms of the combined groups (when considering only carbon atoms of non-aromatic groups while disregarding carbon atoms of aromatic groups) does not exceed 12, wherein each of the above-mentioned groups specified for R₄ may optionally be further substituted by 1-3 substituents independently selected from halogen, methyl, hydroxy and methoxy; each M is a divalent group independently selected from the group consisting of C₁₋₄ alkylene, C₂₋₄ alkenylene, C₂₋₄ alkynylene and a covalent bond, each L is a divalent group independently selected from the group consisting of —(CO)—, —O—, —COO—, —OCO—, and a covalent bond, with the proviso that adjacent M and L groups cannot both be a covalent bond at the same time. Preferably, R₄ is selected from the group consisting of linear C₁₋₆ alkyl, branched C₃₋₆ alkyl, cyclic C₃₋₆ alkyl, and linear C₂₋₆ alkenyl. In a preferred aspect, the substituent group represented by one of the formulae -M-L-R₄, -L-M-R₄, -L-M-L-R₄ and -M-L-M-R₄ has a total number of carbon atoms ranging from 1 to 12, more preferably 1 to 7.

In yet another aspect, R₂ is selected according to any one of specific Aspects (i), (ii) and (iii) as defined in the following while R₁ is as specified above.

According to Aspect (i), R₂ is selected from C₁₋₄-alkyl groups such as methyl, ethyl, propyl, isopropyl, butyl, isobutyl, and t-butyl.

According to Aspect (ii), R₂ is selected from C5 or C6 alkyl groups such as pentyl, isopentyl, hexyl, and isohexyl, C2-4-alkenyl groups such as 2-propenyl, allyl, crotyl, 1-butenyl, 2-butenyl, 3-buten-1-yl, butadienyl, cyclic C3-10-alkyl groups such as cyclopropylmethyl, cyclopentyl, cyclohexyl, cycloheptyl, adamantyl and 1-cyclohexylpropyl, and C1-3-alkyl-C6-14-aryl such as benzyl.

According to Aspect (iii), R₂ is selected from
C7 or C8 alkyl groups such as heptyl, isoheptyl, octyl, and isooctyl,
C2-4-alkynyl groups such as ethynyl, 1-propynyl, 2-propynyl, 1-butynyl, 2-butynyl, 3-butynyl and propagyl,
C6-14-aryl groups such as phenyl, naphthyl, anisyl, toluyl, and xylenyl,
C6-14-aryl-C1-6-alkyl,
heteroaryl and non-aromatic heterocyclic groups each being monocyclic or polycyclic and having 5 to 14 ring members and 1 to 4 heteroatoms selected from N, S and O, such as pyrimidine, morpholine, piperazine, piperidine, tetrahydropyranyl, and thiophene, a substituted group wherein the substituted group is selected from C1-8-alkyl groups, C2-4-alkenyl groups, and C6-14-aryl groups, each of which carrying 1 to 4 substituents independently selected from fluorine, chlorine, C6-14-aryl, which itself optionally carries 1 or 2 substituents selected from halogen and C1-2-alkoxy, linear, branched or cyclic C1-6-alkyl, C1-4-alkoxy, C1-4-alkoxy-C1-2-alkyleneoxy, hydroxy-C1-4-alkoxy, hydroxy-C1-4-alkoxy-C1-2-alkyleneoxy, C1-6-alkyl-CO—O—, C6-10-aryl-C1-2-alkyl-CO—O—, heteroaryl and non-aromatic heterocyclic groups each being monocyclic or polycyclic and having 5 to 14 ring members and 1 to 4 heteroatoms selected from N, S and O, and C6-14-aryl-carbonyl, which itself optionally carries 1 or 2 substituents selected from halogen and C1-2-alkoxy, such as 1-cyclohexylpropyl, fluoromethyl, 1-fluoroethyl, 2-fluoroethyl, difluoromethyl, trifluoromethyl and pentafluoroethyl, chlorodimethyl, chloromethyl, 2-chloroethyl, 2,4-dichlorophenyl, 1,1,2,2-tetrachloroethyl, 1-chlorobutyl, and 4-chlorobenzyl, 9-fluorenylmethyl, methoxyethoxymethyl, pivalyloxymethyl, phenylacetoxymethyl, phenacyl and substituted phenacyl such as p-bromophenacyl, p-methoxyphenacyl, and also 3-methyl-3-pentyl, cinnamyl, oxazol-2-yl, oxazol-4-yl, and 2-C₁₋₄ alkyl-1,3-oxazolin-4-yl, triphenylmethyl, p-methoxybenzyl, 4-picolyl, diphenylmethyl, phenylethyl, substituted phenylethyl, but also alkoxyalkyl such as methoxymethyl, ethoxymethyl, propoxymethyl, butoxymethyl, methoxyethyl, ethoxyethyl, propoxyethyl, isopropoxyethyl, butoxyethyl, isobutoxyethyl, hydroxyalkoxyalkyl such as hydroxymethoxymethyl, 2-hydroxyethoxymethyl, 3-hydroxypropoxymethyl, 4-hydroxybuthoxymethyl, hydroxymethoxyethyl, hydroxymethoxypropyl hydroxymethoxybutyl, hydroxymethoxypentyl, hydroxymethoxyhexyl, polyhydroxyalkyl, and hydroxypolyalkyleneoxyalkyl and also carbonyloxylalkyl such as acetoyloxymethyl, propanoyloxymethyl, butanoyloxymethyl, pentanoyloxymethyl, hexanoyloxymethyl, and pivaloyloxymethyl.

R₂ is preferably selected from the groups listed above as groups of aspects (i) and (ii), and more preferably selected from the groups of aspect (i).

R₂ preferably comprises 1 or more substituent selected from a halogen atom, —OH and a C₁₋₄ alkoxy group. R₂ more preferably comprises methyl, ethyl, propyl, isopropyl, butyl. R₂ is particularly preferably selected from methyl and ethyl.

The total number of carbon atoms in the compound represented by Formula I is preferably from 7 to 69. In one preferred aspect, the total number of carbon atoms in the compound represented by Formula I is from 7 to 30, more preferably from 15 to 23.

In a preferred aspect, the compound represented by general Formula I is selected from compounds represented by formulae A, B, C, D and E.

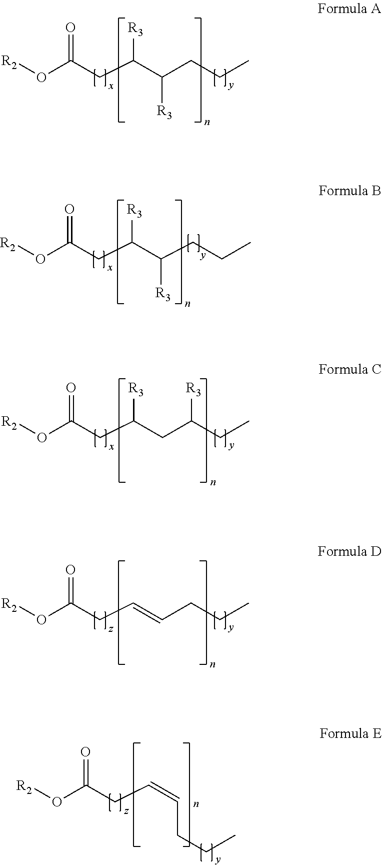

In each of above Formulae A to E, n is an integer of 0-6, x=0-15, preferably 1-7, y=0-15, preferably 1-7, z=1-15, preferably 1-7, with the provision that the total number of carbon atoms in each of Formulae A, B, C, D and E (including the carbon atom that is part of the carbonyl group but excluding the carbon atoms contained in R₂) is 16 or more and 22 or less, preferably 16 to 18; and each R₃ is independently selected from H or I, with the provisions that the total number of iodine atoms represented by R₃ is 0 to 6, and that the iodine atoms are neither geminal nor vicinal; and where R₂ has the same meaning as specified above in relation to formula I.

In one aspect, examples of suitable R₂ groups include, but are not limited to, unsubstituted alkyl, such as methyl, ethyl, propyl, isopropyl, butyl, isobutyl, pentyl, isopentyl, hexyl, isohexyl, heptyl, isoheptyl, octyl, isooctyl and similar but also substituted alkyl groups such as 9-fluorenylmethyl, methoxyethoxymethyl, tetrahydropyranyl, pivaloyloxymethyl, phenylacetoxymethyl, phenacyl and substituted phenacyl such as p-bromophenacyl, p-methoxyphenacyl, and also t-butyl, 3-methyl-3-pentyl, cyclopentyl, cyclohexyl, allyl, 3-buten-1-yl, cinnamyl, oxazole, and 2-alkyl-1,3-oxazoline. It also includes alkylaryl such as benzyl, substituted benzyl such as triphenylmethyl, p-methoxybenzyl, 4-picolyl, diphenylmethyl phenylethyl, substituted phenylethyl, but also alkoxyalkyl such as methoxymethyl, ethoxymethyl, propoxymethyl, butoxymethyl, methoxyethyl, ethoxyethyl, propoxyethyl, isopropoxyethyl, butoxyethyl, isobutoxyethyl, hydroxyalkoxyalkyl such as hydroxymethoxymethyl, 2-hydroxyethoxymethyl, 3-hydroxypropoxymethyl, 4-hydroxybuthoxymethyl, hydroxymethoxyethyl, hydroxymethoxypropyl hydroxymethoxybutyl, hydroxymethoxypentyl, hydroxymethoxyhexyl, polyhydroxyalkyl, hydroxypolyalkyleneoxyalkyl, and also carbonyloxylalkyl such as acetoyloxymethyl, propanoyloxymethyl, butanoyloxymethyl, pentanoyloxymethyl, hexanoyloxymethyl, pivaloyloxymethyl. R₂ is preferably selected from methyl, ethyl, propyl, isopropyl, butyl, and isobutyl, more preferably methyl or ethyl.

According to the invention, the ester component defined herein contains neither one or more ethyleneoxy groups nor a mono- or disaccharide group such as a sucrose group, nor a sugar alcohol group.

According to another aspect, it is possible to use a mixture of two or more ester compounds as defined hereinabove.

Definition E2: The ester component is as defined above in accordance with Definition E1, however with the proviso that the R₁ group does not carry any iodine atoms. That is, iodine is excluded from the substituents possible for R₁ of formula I. Moreover, in formulae A, B and C, R₃ cannot be iodine. In some specific aspects, the ester component of definition E2 does not carry any iodine atom in any position, i.e. neither R₁ nor R₂ contains any iodine atoms.

Definition E3: The ester component is represented by formula I as shown above as part of Definition E1. The R₁ group is a linear C₁₃-C₁₉ alkyl group or a linear C₁₃-C₁₉ alkenyl group, each of which may optionally be substituted with 1, 2 or 3 halogen atoms selected from I, F, Cl and Br. Preferably, the R₁ group is a linear C₁₅-C₁₇ alkyl group or a linear C₁₅-C₁₇ alkenyl group, each of which may optionally be substituted with 1, 2 or 3 halogen atoms selected from F, Cl and Br. That is, for this preferred embodiment, iodine is excluded from the substituents possible for R₁ of formula I. This means that R₁ can be as defined in formulae A, B, C, D and E, with the provisos that x, y, z and n are selected such that the total number of carbon atoms in each of Formulae A, B, C, D and E (including the carbon atom that is part of the carbonyl group but excluding the carbon atoms contained in R₂) is 14-20 and preferably 16-18, and that R₃ cannot be iodine. The R₂ group is selected from linear or branched C₁-C₆ alkyl groups or linear or branched C₁-C₆ alkenyl groups and preferably linear or branched C₁-C₄ alkyl groups or linear or branched C₁-C₄ alkenyl groups, each of which may be unsubstituted or carry 1 or 2 substituents independently selected from F, Cl, Br, I, OH and —OCH₃, preferably independently selected from F, Cl, and —OCH₃. A mixture of two or more ester compounds according to this definition E3 may also be used.

Definition E4: The ester component is as defined above in accordance with Definition E3, however with the proviso that the R₁ group does not carry any iodine atoms. That is, iodine is excluded from the substituents possible for R₁ of formula I. Moreover, in formulae A, B and C, R₃ cannot be iodine. In some specific aspects, the ester component of definition E4 does not carry any iodine atom in any position, i.e. neither R₁ nor R₂ contains any iodine atoms.

### Water Component

Definition W1: The aqueous phase consists of or contains water as an essential constituent. All different grades and purities of water may be used. Of course, if the emulsion is intended for administration to humans, water must be used, which is suitable for human consumption. For example, tap water, mineral water and purified water (distilled water, bi-distilled water, reverse osmosis water, milliQ water) may be used.

Additional components may optionally be present. The water component forming the aqueous phase may contain one or more water-soluble substances and/or one or more solid substances that can be dispersed in water, such as solid components of unfiltered fruit juice in addition to water. It is also possible to use an aqueous liquid. For example, if the emulsion is intended for oral administration to humans, it is possible to use a water-based beverage such as carbonated water, soft drinks (carbonated, flavoured and typically sweetened beverages such as cola, lemonade or ginger ale), fruit juice, coffee, tea, milk or plant-based milk alternative. Alternatively, pharmaceutical grade aqueous solutions such as pharmaceutically acceptable buffers (citrate, borate, phosphate, carbonate, acetate), saline, glucose, sucrose, mannitol or sorbitol solution may be used.

Preferred water components are drinking water such as tap water, mineral water, spring water, or carbonated water, coffee, tea, milk, fruit juice, and soft drinks. More preferred water components are tap water, spring water, mineral water, coffee, and tea.

However, if a water component other than pure water is used, the weight of additional components, e.g. flavouring compounds, sweeteners, etc., which are introduced as part of the water component, is not to be treated as weight contributing to the weight of the water component (referred to as w % hereinbelow) so that only the weight of the water itself is to be considered as weight of the water component (referred to as w % hereinbelow). For example, if a fruit juice with 90 wt % water and 10 wt % further components (e.g. pulp and/or sugar) is used, only the 90 wt % of the water will be considered when determining the weight of the water component, whereas the weight of the further fruit juice components is disregarded.

Components that are an integral part of beverages used as water component (such as for example minerals, sweeteners, sugars, vitamins, polyphenols, caffeine, flavonoids, flavours, amino acids, colouring agents, proteins, milk fat, fruit pulp or any other component originating from soft drinks, fruit juice, tea, coffee, milk or any other beverage) are also not to be considered when determining the weight of the optional components.

In an alternative aspect of characterizing the invention, the weight of such beverage components is considered when determining the relative content of the optional further component, as specified in definition O1 or O2 below. However, when applying this alternative aspect of characterizing the invention, total and individual relative amounts of the optional further components may be up to 10 wt. % higher than specified below. For example, the upper limit for the broadest range in definition O1 would be 15 wt. % instead of 5 wt. % and the upper limit for the definition O2 would be 10 wt. % instead of 0 wt. % (while the lower limits would remain the same). Of course, amount indications for the premix remain the same since the premix does not contain a water component.

Definition W2: The aqueous phase is formed by a water-based beverage other than purified water as such, such as tap water, mineral water, carbonated water, soft drinks (carbonated, flavoured and typically sweetened beverages such as cola, lemonade or ginger ale), fruit juice, coffee, tea, milk or pharmaceutical grade aqueous solutions such as pharmaceutically acceptable buffers (e.g. citrate, borate, phosphate, carbonate, acetate), saline, sucrose, glucose, mannitol or sorbitol solution.

Preferred water components are drinking water such as tap water, mineral water, spring water, or carbonated water, coffee, tea, milk, fruit juice, and soft drinks. More preferred water components are tap water, spring water, mineral water, coffee, and tea.

The treatment of the weight of additional components of beverages is the same as described above for definition W1.

### Surfactant

Definition S1: The surfactant used in the present invention is not particularly limited, as long as it is a non-ionic surfactant. This means, of course, that the only surfactant used in the present invention is one or more non-ionic surfactants. The surfactant preferably is a single surfactant. It may include compounds containing at least one hydrophilic group, preferably selected from polyoxyethylene groups having from 3-60, preferably 4-40, ethyleneoxy units, monosaccharides such as sucrose, disaccharides, sugar alcohols and combinations thereof, as well as at least one hydrophobic group containing a saturated or unsaturated hydrocarbon group, preferably alkyl group, said hydrocarbon group having 8-20, preferably 12-18, carbon atoms. Preferably, the surfactant is represented by the following general formula (S), (S1), (S2), (S3), (S4) or (S5):

(HPI)ₙL(IPO)ₘ (S)

(HPI—HIPO)ₙ₁L(HIPO)ₘ₁ (S1)

(HPI—HIPO—HAPI)ₙ₂L(HIPO)ₘ₂ (S2)

(HPI—HIPO)ₙ₃L(HIPI)ₘ₃ (S3)

(HPI—HIPO—HAPI)ₙ₄L(HIPI)ₘ₄ (S4)

(HPI)—(HPA)-(HPI) (S5)
wherein
HPI represents a group selected from a polyoxyethylene group having from 3-60, preferably 4-40, ethyleneoxy units, a mono- or disaccharide, a sugar alcohol having from 5-6 carbon atoms or a combination thereof, wherein, in case of a combination of two or more, preferably 2-5 of these groups, the groups are bonded to each other via a functional group independently selected from an ester group, a carbonate group, and an ether group;
L represents a moiety derived from glycerol or is absent;
HPO represents a hydrocarbon group having 8-20, preferably 12-18, carbon atoms, preferably an alkyl or alkenyl group having 8-20, preferably 12-18, carbon atoms, optionally carrying a hydroxyl substituent; and
wherein HPI is bonded to L and/or HPO via a functional group independently selected from an ester group, a carbonate group, and an ether group, preferably an ester group; wherein L is bonded to HPO via a functional group independently selected from an ester group, a carbonate group, and an ether group, preferably an ester group; wherein the monosaccharide, disaccharide and sugar alcohol groups may be bonded to the adjacent groups via any of the oxygen atoms that form hydroxyl groups in the free form; and wherein each of the polyoxyethylene, monosaccharide, disaccharide and sugar alcohol groups may be present in any orientation, with the proviso that compounds with unstable groups, like peroxy groups or perester groups, are not meant to be encompassed.
n is 0, 1 or 2; m is 1 or 2; with the provisos that n+m≤3 if L is glycerol and n+m=2 if L is absent;
n1 is 1, 2 or 3; m1 is 0, 1 or 2; with the provisos that L is glycerol and n1+m1≤3;
n2 is 1, 2 or 3; m2 is 0, 1 or 2; with the provisos that n1+m1≤3 if L is glycerol and n2+m2=2 if L is absent;
n3 is 1, 2 or 3; m3 is 0, 1 or 2; with the provisos that n3+m3≤3 if L is glycerol and n3+m3=2 if L is absent;
n4 is 1, 2 or 3; m4 is 0, 1 or 2; with the provisos that L is glycerol and n4+m4≤3; and, wherein in formula (S5), HPI represents a polyoxyethylene group having from 2-130, preferably 3-60, more preferably 4-40, ethyleneoxy units and HPA represents a polyoxypropylene group having from 15-67, preferably 20-60, more preferably 25-40, propyleneoxy units. Compounds of formula (S5) may also be represented by the following general formula HO—(CH₂CH₂—O—)ₐ—(CH(CH₃)—CH₂—O—)_{b}—(CH₂CH₂—O—)ₐ—H, with a ranging from 2 to 167 and b ranging from 15 to 67. These compounds are also referred to herein as poloxamers.

If L is glycerol, each of HPI and HPO is bonded to L via a functional group independently selected from an ester group, a carbonate group, and an ether group.

If L is absent, HPI and HPO are bonded to each other via a functional group independently selected from an ester group, a carbonate group, and an ether group. In formulae (S1), (S2), (S3), (S4), and (S5), in the moieties (HPI—HPO) and (HPI—HPO—HPI), HPI and HPO are bonded to each other via a functional group independently selected from an ester group, a carbonate group, and an ether group.

More preferably, the surfactant is selected from a group consisting of (S11) polyoxyethylene castor oils wherein the total number of oxyethylene units is preferably 4 or 5 or any higher number up to 60 or even up to 200, (S12) polyoxyethylene sorbitan fatty acid esters wherein the total number of oxyethylene units is preferably 4, 5 or any higher number up to 20 and the fatty acid is preferably a fatty acid having 12 (e.g. lauric acid) to 18 (e.g. stearic or oleic acid) carbon atoms, which may be saturated or unsaturated, (S13) macrogol 15 hydroxystearate (i.e. a product obtained by reacting 15 mol ethyleneoxide with 1 mol 12-hydroxystearic acid), (S14) polyoxyethylene alkyl ethers wherein the total number of oxyethylene units is preferably 4 or 5 or any higher number up to 25 and wherein the alkyl group of the alkyl ether preferably has from 12 to 18 carbon atoms, (S15) sucrose fatty acid esters wherein a sucrose moiety may typically carry 1, 2 or 3 fatty acid ester groups, each of which being preferably derived from fatty acids having has 12-20 carbon atoms, more preferably 16-18 carbon atoms, (S16) caprylocaproyl polyoxylglycerides, also known as caprylocaproyl macrogolglycerides, (S17) poloxamers of formula (S5), and (S18) monoglycerides of fatty acids, wherein the fatty acid is preferably a fatty acid having 12 (e.g. lauric acid) to 18 (e.g. stearic or oleic acid) carbon atoms, which may be saturated or unsaturated such as glyceryl monostearate, glyceryl monolinoleate and glyceryl monooleate.

Polyethylene castor oils can be represented by the following general formulas:

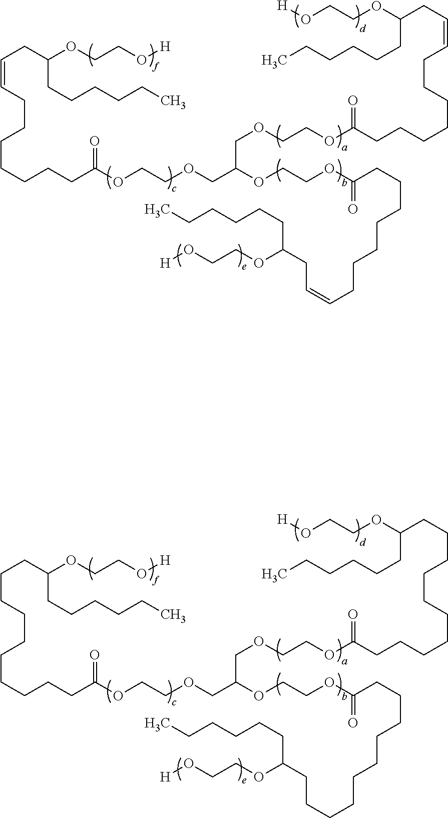

wherein each of a, b, c, d, e, f is independently selected from the range of 0 to 35. In one aspect, the polyethylene castor oil is a mixture of triricinoleate esters of ethoxylated glycerol with small amounts of polyethylene glycol ricinoleate and the corresponding free glycols having typically 30 to 50 ethylene oxide units. In another aspect, the polyethylene castor oil is a mixture of trihydroxystearate esters of ethoxylated glycerol with small amounts of macrogol trihydroxystearate and the corresponding free glycols, having typically 40-60 ethylene oxide units.

More preferably, the surfactant is selected from a group consisting of polyoxyethylene castor oils wherein the total number of oxyethylene units is preferably 4 or 5 or any higher number up to 60 and polyoxyethylene sorbitan fatty acid esters wherein the total number of oxyethylene units is preferably 4, 5 or 20 and the fatty acid is preferably a fatty acid having 12 (lauric acid) to 18 (stearic or oleic acid) carbon atoms, which may be saturated or unsaturated.

(S11a) The polyoxyethylene castor oils may be selected from, but are not limited to, polyoxyl 5 castor oil (Acconon CA-5; castor oil POE-5; Etocas 5; Hetoxide C-5; Jeechem CA-5; PEG-5 castor oil; polyoxyethylene 5 castor oil), polyoxyl 9 castor oil (Acconon CA-9; castor oil POE-9; Jeechem CA-9; PEG-9 castor oil; polyoxyethylene 9 castor oil; Protachem CA-9), polyoxyl 15 castor oil (Acconon CA-15; castor oil POE-15; Jeechem CA-15; PEG-15 castor oil; polyoxyethylene 15 castor oil; Protachem CA-15), polyoxyl 35 castor oil (Castor oil POE-35; Cremophor EL; Cremophor ELP; Etocas 35; glycerol polyethyleneglycol ricinoleate; Kolliphor EL; Kolliphor ELP; PEG-35 castor oil; polyethoxylated castor oil; polyoxyethylene 35 castor oil), polyoxyl 40 castor oil (Castor oil POE-40; Cirrasol G-1284; Croduret 40; Etocas 40; Eumulgin RO; Hetoxide C40; Jeechem CA-40; Kolliphor RH40; Marlowet R40; Nikkol CO 40TX; Nonionic GR-40; PEG-40 castor oil; polyoxyethylene 40 castor oil; Protachem CA-40), polyoxyl 40 hydrogenated castor oil (Cremophor RH 40; Croduret 40; Eumulgin HRE 40PH; glycerol polyethyleneglycol oxystearate; Hetoxide HC40; hydrogenated castor oil POE-40; Jeechem CAH-40; PEG-40 hydrogenated castor oil; polyethoxylated hydrogenated castor oil; polyoxyethylene 40 hydrogenated castor oil; Lipocol HCO-40; Lipocol LAV HCO 40; Nikkol HCO 40 Pharma; Nonionic GRH-40; Protachem CAH-40), polyoxyl 60 castor oil (Castor oil POE-60; Jeechem CA-60; Nikkol CO60TX; PEG-60 castor oil; polyoxyethylene 60 castor oil), polyoxyl 60 hydrogenated castor oil (Cremophor RH 60; Croduret 60; Eumulgin HRE 60PH; Hetoxide HC60; hydrogenated castor oil POE-60; Jeechem CAH-60; PEG-60 hydrogenated castor oil; polyoxyethylene 60 hydrogenated castor oil; Lipocol HCO-60; Nikkol HCO 60 Pharma; Protachem CAH-60), polyoxyl 100 castor oil (Hydrogenated castor oil POE-100; Jeechem CA-100; PEG-100 hydrogenated castor oil; polyoxyethylene 100 hydrogenated castor oil), polyoxyl 100 hydrogenated castor oil (Cirrasol G-1300; Jeechem CA-100; Nikkol HCO 100; polyoxyethylene 100 hydrogenated castor oil), polyoxyl 200 castor oil (Hetoxide C200; Jeechem CA-200; polyoxyethylene 200 castor oil; PEG-200 castor oil; castor oil POE-200), polyoxyl 200 hydrogenated castor oil (Hydrogenated castor oil POE-200; Jeechem CAH-200; PEG-200 hydrogenated castor oil; polyoxyethylene 200 hydrogenated castor oil).

(S12a) The polyoxyethylene sorbitan fatty acid esters may be selected from, but are not limited to, polyoxyethylene 20 sorbitan monolaurate (polysorbate 20) (Alkest TW 20; Armotan PML 20; Capmul POE-L; Campul POE-L Low PV; Crillet 1; Drewmulse; E432; Durfax 20; E432; Eumulgin SML; Glycosperse L -20; Hodag PSML-20; Kolliphor PS 20; Lamesorb SML-20; Liposorb L-20; Liposorb L-20K; Montanox 20; Nissan Nonion LT-221; Norfox Sorbo T-20; POESML; polysorbatum 20; Ritabate 20; Sorbax PML-20; sorbitan monododecanoate; Sorgen TW-20; T-Maz 20; T-Maz 20K; poly(oxy-1,2-ethanediyl) derivatives; polyoxyethylene 20 laurate; Protasorb L-20; Tego SML 20; Tween 20), polyoxyethylene (4) sorbitan monolaurate (polysorbate 21) (Crillet 11; Hodag PSML-4; Protasorb L-5; Tween 21), polyoxyethylene 20 sorbitan monopalmitate (polysorbate 40) (Crillet 2; E434; Eumulgin SMP; Glycosperse S-20; Hodag PSMP-20; Lamesorb SMP-20; Liposorb P-20; Lonzest SMP-20; Montanox 40; poly(oxy-1,2-ethanediyl) derivatives; polysorbatum 40; Protasorb P-20; Ritabate 40; sorbitan monohexadecanoate; Sorbax PMP-20; Tween 40), polyoxyethylene 20 sorbitan monostearate (polysorbate 60) (Alkest TW 60; Atlas 70K; Atlas Armotan PMS 20; Capmul POE-S; Cremophor PS 60; Crillet 3; Drewpone 60K; Durfax 60; Durfax 60K; E435; Emrite 6125; Eumulgin SMS; Glycosperse S-20; Glycosperse S-20FG; Glycosperse S-20FKG; Hodag PSMS-20; Hodag SVS-18; Kolliphor PS 60; Lamsorb SMS-20; Liposorb S-20; Liposorb S-20K; Lonzest SMS-20; Montanox 60; Nikkol TS-10; Norfox SorboT-60; Polycon T 60 K; polyoxyethylene 20 stearate; polysorbatum 60; Protasorb S-20; Ritabate 60; Sorbax PMS-20; sorbitan monooctadecanoate poly(oxy-1,2-ethanediyl) derivatives; T-Maz 60; T-Max 60KHS; Tween 60; Tween 60K; Tween 60 VS), polyoxyethylene 20 sorbitan tristearate (polysorbate 65) (Alkamuls PSTS-20; Crillet 35; E436; Glycosperse TS-20; Glycosperse TS-20 FG; Glycosperse TS-20 KFG; Hodag PSTS-20; Lamesorb STS-20; Lanzet STS-20; Liposorb TS-20; Liposorb TS-20A; Liposorb TS-20K; Montanox 65; Protasorb STS-20; Sorbax PTS-20; sorbitan trioctadecanoate poly(oxy-1,2-ethanediyl) derivatives; T-Maz 65K; Tween 65; Tween 65K; Tween 65V), polyoxyethylene 20 sorbitan monooleate (polysorbate 80) (Alkest TW 80; Atlas E; Armotan PMO 20; Capmul POE-O; Cremophor PS 80; Crillet 4; Crillet 50; Drewmulse POE-SMO; Drewpone 80K; Durfax 80; Durfax 80K; E433; Emrite 6120; Eumulgin SMO; Glycosperse 0-20; Hodag PSMO-20; Kolliphor PS 80; Liposorb 0-20; Liposorb 0-20K; Montanox 80; polyoxyethylene 20 oleate; polysorbatum 80; Protasorb 0-20; Ritabate 80; (Z)-sorbitan mono-9-octadecenoate poly(oxy1,2-ethanediyl) derivatives; Tego SMO 80; Tego SMO 80V; Tween 80), polyoxyethylene (5) sorbitan monooleate (polysorbate 81) (Crillet 41; Hetsorb 0-5; Hodag PSMO-5; Protasorb 0-5; Sorbax PMO-5; sorbitan mono-9-octadecenoate poly(oxy-1,2-ethanediyl) derivatives; T-Maz 81; Tego SMO 81; Tween 81), polyoxyethylene 20 sorbitan trioleate (polysorbate 85) (Alkamuls PSTO-20; Crillet 45; Glycosperse TO-20; Hodag PSTO-20; Liposorb TO-20; Lonzest STO-20; Montanox 85; Protasorb TO-20; Sorbax PTO-20; sorbitan tri-9-octadecenoate poly(oxy1,2-ethanediyl) derivatives; Tego STO 85; Tween 85), and polyoxyethylene 20 sorbitan monoisostearate (polysorbate 120) (Crillet 6).

(S13a) The macrogol 15 hydroxystearate may be selected from, but is not limited to, polyoxyl 15 hydroxystearate, Kolliphor HS 15, and Solutol HS 15.

(S14a) The polyoxyethylene alkyl ethers may be selected from, but are not limited to, Cetomacrogol 1000 (Polyethylene glycol 1000 macrocetyl ether; polyoxyethylene glycol 1000 monocetyl ether; Cresmer 1000), polyoxyl 6 cetostearyl ether (Ceteareth 6; Cremophor A6; Volpo CS6), polyoxyl 20 cetostearyl ether (Atlas G-3713; Ceteareth 20; Cremophor A 20 polyether; Volpo CS20), polyoxyl 25 cetostearyl ether (Ceteareth 25; Cremophor A25; Volpo CS25), polyoxyl 2 cetyl ether (BC-2; Brij 52; ceteth-2; Lipocol C-2; Procol CA-2), polyoxyl 10 cetyl ether (BC-10TX; Brij 56; ceteth-10; Lipocol C-10; Procol CA-10), polyoxyl 20 cetyl ether (BC-20TX; Brij 58; ceteth-20; Lipocol C-20; Volpo C20), polyoxyl 4 lauryl ether (BL-4.2; Brij 30; laureth-4; Lipocol L-4; Procol LA-4; Tego Alkanol L4; Volpo L4), polyoxyl 9 lauryl ether (BL-9EX; laureth-9; lauromacrogol 400; polidocanol; Volpo L9), polyoxyl 23 lauryl ether (Brij 35; laureth-23; Lipocol L-23; Procol LA-23; Ritox 35; Tego Alkanol L23 P), polyoxyl 2 oleyl ether (BO-2V; Brij 92; Brij 93; oleth-2; Lipocol 0-2; Procol OA-2; Ritoleth 2; Volpo N2), polyoxyl 10 oleyl ether (BO-10V; Brij 96; Brij 97; oleth-10; polyethylene glycol monooleyl ether; Lipocol O-10; Procol OA-10; Ritoleth 10; Volpo N10), polyoxyl 20 oleyl ether (BO-20V; Brij 98; Brij 99; Lipocol 0-20; oleth-20; Procol OA-20; Ritoleth 20; Volpo N20), polyoxyl 2 stearyl ether (BS-2; Brij 72; Lipocol S-2; Procol SA-2; steareth-2; Tego Alkanol S2; Volpo S-2), polyoxyl 10 stearyl ether (Brij 76; Lipocol S-10; Procol SA-10; steareth-10; Tego Alkanol 510; Volpo 5-10), polyoxyl 21 stearyl ether (Brij 721; Ritox 721; Steareth-21), and polyoxyl 100 stearyl ether (Brij 700; steareth-100).

(S15a) The sucrose esters (sucrose fatty esters) may be selected from, but are not limited to, sucrose laurate, sucrose myristate, sucrose palmitate and sucrose stearate.

(S16a) The caprylocaproyl polyoxylglycerides may be selected from, but are not limited to, DUBCARE GPE 810, Labrasol, Labrasol ALF, macrogolglyceridorum caprylocaprates, PEG 400 caprylic/capric glycerides.

(S17a) The poloxamers may be selected from, but are not limited to, Poloxamer 407 (Pluronic F-127, Kolliphor P 407), Poloxamer 188 (Pluronic F-68, Kolliphor P 188) or Poloxamer 124 (Pluronic L44, Kollisolv P 124). Poloxamer 124 is most preferred.

(S18a) The monoglycerides of fatty acids may be selected from glyceryl monostearate, glyceryl monopalmitate, glyceryl arachidate, glyceryl monolinoleate and glyceryl monooleate, most preferably glyceryl monostearate. Glyceryl monostearate can be the pure isomer 1-glyceryl monostearate, the pure isomer 2-glyceryl monostearate, or a mixture of both. The same applies also to other glycerol monoester of fatty acids: the fatty acid may be bonded to the hydroxyl group at C1 or at C2 or be a mixture of both.

It is also possible to use a combination of two or more surfactants wherein each of the surfactants falls within the definition Si. Even more preferably, the surfactant according to definition S1 is used as a single surfactant, i.e. as the only surfactant, wherein neither a second surfactant falling within the definition S1 nor any other surfactant is present in addition to the surfactant of definition S1.

Definition S2: The surfactant is selected from polyoxyethylene castor oils wherein the total number of oxyethylene units is preferably 4 or 5 or any higher number up to 60 or even up to 200, polyoxyethylene sorbitan fatty acid esters wherein the total number of oxyethylene units is preferably 4, 5 or any higher number up to 20 and the fatty acid is preferably a fatty acid having 12 (e.g. lauric acid) to 18 (e.g. stearic or oleic acid) carbon atoms, which may be saturated or unsaturated, poloxamers and monoglycerides of fatty acids. The polyoxyethylene castor oil or polyoxyethylene sorbitan fatty acid ester is preferably selected from the specific surfactants listed in the corresponding groups (S11a) and (S12a) above. The poloxamers and monoglycerides of fatty acids are preferably selected from the specific surfactants listed in the corresponding groups (S17a) and (S18a) above.

It is also possible to use a combination of two or more surfactants wherein at least one of the surfactants falls within the definition S2, preferably wherein each of the surfactants falls within the definition S2. Even more preferably, the surfactant according to definition S2 is used as a single surfactant, i.e. as the only surfactant, wherein neither a second surfactant falling within the definition S2 nor any other surfactant is present in addition to the surfactant of definition S2.

Definition S3: The surfactant is selected from polyoxyethylene castor oils wherein the total number of oxyethylene units is preferably 4 or 5 or any higher number up to 60 or even up to 200, polyoxyethylene sorbitan fatty acid esters wherein the total number of oxyethylene units is preferably 4, 5 or any higher number up to 20 and the fatty acid is preferably a fatty acid having 12 (e.g. lauric acid) to 18 (e.g. stearic or oleic acid) carbon atoms, which may be saturated or unsaturated. The polyoxyethylene castor oil or polyoxyethylene sorbitan fatty acid ester is preferably selected from the specific surfactants listed in the corresponding groups (S11a) and (S12a) above.

It is also possible to use a combination of two or more surfactants wherein at least one of the surfactants falls within the definition S3, preferably wherein each of the surfactants falls within the definition S3. Even more preferably, the surfactant according to definition S3 is used as a single surfactant, i.e. as the only surfactant, wherein neither a second surfactant falling within the definition S3 nor any other surfactant is present in addition to the surfactant of definition S3.

### Optional Further Components

Definition O1: One or more further components may optionally be present. The optional further component may for instance be selected from flavouring agents, colouring agents, stabilizing agents, or it may be any other component that provides desired functionality or properties to the emulsion of the invention. In one aspect, one or more triglycerides derived from C₁₄₋₂₀ fatty acids is/are present as optional further component(s).

Such further optional components are not particularly restricted in terms of type and amount as long as there is no significant impact on the properties of the final emulsion. This can be tested by preparing two emulsions in analogy to Example 10 below, which differ from each other only with respect to the presence or absence of the optional further component of interest. There is no significant impact if the particle size of the sample with optional component differs by less than 10% from the particle size of the sample without optional component immediately after preparation and differs by less than 15% from the particle size of the sample without optional component after storage of both samples for 1 month at 5° C.

In a preferred embodiment that can be combined with any other embodiment described herein, ethanol is not present in the emulsion, composition for emulsion and premix of the invention.

In another preferred embodiment, no hydrophilic co-solvent is present. This means that all hydrophilic organic compounds that are liquid at 25° C. are excluded. As used herein, a hydrophilic organic compound is to be understood as having a solubility in water of 33 g/L or more at 25° C. Solubility can be determined using the procedure of the European Pharmacopoeia 10^{th} edition, chapter 5.11. The examples of the hydrophilic organic compounds include, but are not limited to, propylene glycol, butylene glycol, glycofurol (tetrahydrofurfuryl alcohol polyethyleneglycol ether), glycerol, acetone, propanol, isopropanol, dimethylacetylamide, dimethylsulfoxide, dimethyl isosorbide, polyethylene glycol, propylene carbonate or benzyl alcohol.

In a preferred embodiment, at least one optional further component is present, which is soluble in the water component. In this connection, the term “soluble in the water component” means that the solubility in the water component must be sufficiently high that the entire amount of the optional further component can completely dissolve in the water component. This can be determined by mixing the optional further component and the pure water component in the same amounts in which they will be used in the emulsion of the invention.

In another preferred embodiment, at least one optional further component is present, which is liquid and miscible with the ester component. In this connection, the term “liquid and miscible with the ester component” means that the optional further component is liquid at room temperature (e.g. 22° C.) and that its entire amount can mix with the ester component such that a single homogenous phase is formed. This can be determined by mixing the optional further component and the pure ester component in the same amounts in which they will be used in the emulsion of the invention.

Definition O2: According to another aspect, the compositions of the present invention do not contain any optional further components, i.e. no components in addition to those specified hereinabove and below as essential components.

### First Embodiment

### Components to be Used

There is no particular restriction regarding the components to be used in the first embodiment. That is, an ester component can be selected from the ester components according to anyone of definition E1, E2, E3 or E4. Likewise, a water component can be a use according to anyone of definitions W1 and W2. A surfactant can be used according to anyone of definitions S1, S2 and S3. The indications of preference provided above apply to the components to be used in the first embodiment.

In addition to the above-mentioned essential components, it is possible to use one or more optional further components in accordance with definition 01 or to omit such optional further components in accordance with definition 02.

### Relative Amounts of Components

In an embodiment of the invention, the weight of ester component in relation to the total weight of ester component, surfactant and water in the emulsion of the invention is more than 0%, such as 0.01% or more, and 60% or less and more specifically between 1 and 55%.

In a preferred embodiment of the present invention, the weight of ester component in relation to the total weight of ester component, surfactant and water in the emulsion of the invention is between 5 and 50%.

In a more preferred embodiment, the weight of ester component in relation to the total weight of ester component, surfactant and water in the emulsion of the invention is between 10 and 40%.

The above conditions apply equally to the composition for forming the emulsion of the invention.

In an embodiment of the invention, the weight of surfactant in relation to the total weight of ester component, surfactant and water in the emulsion of the invention is more than 0% and 40% or less.

In a preferred embodiment of the present invention, the weight of surfactant in relation to the total weight of ester component, surfactant and water in the emulsion of the invention is more than 0% and 30% or less and preferably between 0.01% and 28%.

In a more preferred embodiment, the weight of surfactant in relation to the total weight of ester component, surfactant and water in the emulsion of the invention is between 4 and 26%. The content of water follows as a direct consequence from the relative amounts of ester component and surfactant based on the formula w %=100%−e %−s %, wherein w % is the percentage of weight of water relative to the total weight of ester component, surfactant and water in the emulsion of the invention, e % is the percentage of weight of ester component relative to the total weight of ester component, surfactant and water in the emulsion of the invention and s % is the percentage of weight of surfactant relative to the total weight of ester component, surfactant and water in the emulsion of the invention. The above indications are valid irrespective whether optional further components are present or absent.

The above conditions apply equally to the composition for forming the emulsion of the invention.

In another aspect of the present invention, the ester component-to-surfactant ratio is between 90:10 and 40:60 (w/w), and the water-to-surfactant ratio is between 1000:1 and 55:45 (w/w) and the water-to-ester component ratio is between 1000:1 and 4:6 (w/w).

In a preferred aspect of the present invention, the ester component-to-surfactant ratio is between 85:15 and 45:55 (w/w), and the water-to-surfactant ratio is between 400:1 and 65:35 and the water-to-ester component ratio is between 400:1 and 45:55 (w/w).

In an even more preferred aspect of the present invention, the ester component-to-surfactant ratio is between 50:50 and 70:30 (w/w) and the water-to-surfactant ratio is between 100:1 and 80:20 (w/w) and the water-to-ester component ratio is between 100:1 and 1:1 (w/w).

In another aspect of the present invention, the ester component-to-surfactant ratio is between 10:1 and 2:1 (w/w) and the water-to-surfactant ratio is between 98:2 and 50:50, preferably between 95:5 and 55:45, even more preferably between 90:10 and 70:35, and the water-to-ester component ratio is between 10:1 and 1:1.

Further preferred aspects are characterized by the combinations of ratios shown in the following table.

| | ester component- | water-to- | water-to- |
|---|---|---|---|
| Aspect | to-surfactant | surfactant | ester component |
| No. | ratio (w/w) | ratio (w/w) | ratio (w/w) |
| 1 | 40:1 to 40:60 | 1000:1 to 55:45 | 1000:1 to 4:6 |
| 2 | 20:1 to 45:55 | 400:1 to 65:35 | 400:1 to 45:55 |
| 3 | 10:1 to 70:30 | 100:1 to 80:20 | 100:1 to 50:50 |
| 4 | 20:1 to 45:55 | 1000:1 to 55:45 | 1000:1 to 45:55 |
| 5 | 20:1 to 45:55 | 800:1 to 55:45 | 800:1 to 50:50 |
| 6 | 10:1 to 55:45 | 800:1 to 55:45 | 800:1 to 50:50 |
| 7 | 10:1 to 40:60 | 90:10 to 55:45 | 70:35 to 50:50 |
| 8 | 5:1 to 45:55 | 90:10 to 80:20 | 70:35 to 50:50 |
| 9 | 10:1 to 70:30 | 98:2 to 55:45 | 10:1 to 50:50 |
| 10 | 20:1 to 45:55 | 98:2 to 80:20 | 5:1 to 45:55 |
| 11 | 20:1 to 45:55 | 95:5 to 55:45 | 90:10 to 50:50 |
| 12 | 10:1 to 55:45 | 95:5 to 80:20 | 70:30 to 50:50 |
| 13 | 91:9 to 67:33 | 98:2 to 50:50 | 91:9 to 50:50 |
| 14 | 91:9 to 67:33 | 95:5 to 55:45 | 91:9 to 50:50 |
| 15 | 91:9 to 67:33 | 90:10 to 70:35 | 91:9 to 50:50 |

These ranges (and the corresponding ranges elsewhere in the present text) are to be understood as boundary conditions that are to be cumulatively fulfilled. That is, within the scope of the specified ranges, an individual value for one range may be used only if there are suitable values within the other two ranges such that all three boundary conditions are simultaneously fulfilled. Hence, only those combinations of ratios are meant to be disclosed as being in accordance with the invention, which simultaneously fulfil all three wt. %0 or ratio boundary conditions.

These conditions apply to the emulsion of the invention and the composition for forming the emulsion of the invention but not to the premix. In the case of the premix, the amount of water component to be added to the premix prior to use must comply to the boundary conditions specified above.

As far as the premix is concerned, in some aspects, the surfactant is present in a weight of 2 to 60% relative to the total weight of ester component and surfactant. In a preferred aspect, the surfactant is present in a weight of 4 to 55% relative to the total weight of ester component and surfactant. In an even more preferred aspect, the surfactant is present in a weight of 9 to 50% relative to the total weight of ester component and surfactant. In some aspects, the surfactant is present in a weight of 10 to 60%, preferably 15 to 55%, more preferably 30 to 50%, relative to the total weight of ester component and surfactant.

All of the above indications are to be calculated taking the total amount of all surfactant components, i.e. amount of essential surfactant as well as the amount of any optional additional surfactant, into account.

The total content of optional components in the emulsion of the invention is preferably 0 to 5 wt. %, more preferably 0 to 2.5 wt. %, and even more preferably 0 to 1 wt. % based on the total weight of the emulsion. As an additional requirement, the content of each individual optional component is 0 to 1 wt. %, preferably 0 to 0.5 wt. % based on the total weight of the emulsion. As far as the premix is concerned, the total content of optional component present in the premix is preferably 0 to 10 wt. %, preferably 0 to 5% and even more preferably 0 to 2% based on the total weight of the premix. In addition, the content of each individual optional component is 0 to 2 wt. %, preferably 0 to 1 wt. % based on the total weight of the premix.

The above conditions apply equally to the composition for forming the emulsion of the invention.

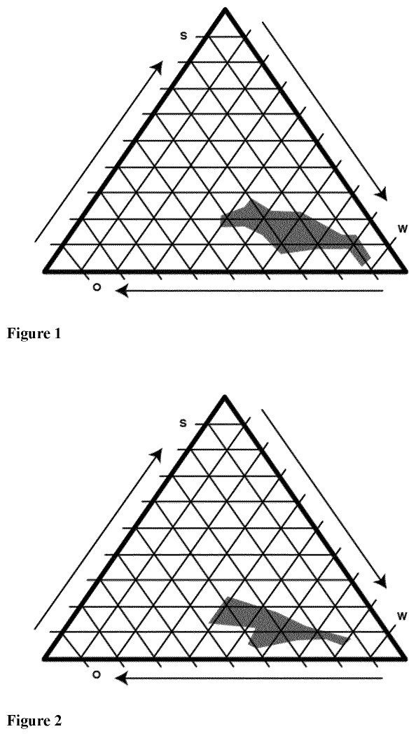

FIGS. 1 to 3 show that not all compositional ratios may be equally suitable. If a particular compositional ratio of interest does not yield the desired emulsion upon shaking or another low-energy manufacturing method, it is possible to determine a suitable compositional ratio in a systematic manner by step-wise changing the relative amounts of ester component, surfactant and water into the direction of a composition composed of 30 wt. % ester component, 55 wt. % water and 15 wt. % surfactant, followed by testing suitability of the composition for forming an emulsion of the invention. For instance, the relative amount of a first one of these components may be suitably increased or decreased in steps of 2 wt. %, 3 wt. % or 5 wt. % and this change may be compensated by decreasing or increasing the amount of one of the other two components by a corresponding amount, or alternatively by decreasing or increasing both other components by smaller amounts such that the total change corresponds to the decrease or increase of the first component.

According to a preferred aspect, the person skilled in the art may select an ester compound according to Formula I and one or more surfactants chosen from polyoxyethylene castor oil derivatives and/or polyoxyethylene sorbitan fatty acid esters and add the selected one or more surfactants to the ester compound of Formula I in a 2:1 ester compound:surfactant ratio and mix. The skilled person may then add water (or an aqueous beverage) at a 2:1 water:ester compound (w/w) ratio and shake for 30 s.

### Emulsion

The emulsion of the first embodiment of the present invention is characterized by the presence of the above-specified components. These components are present in relative amounts as specified hereinabove for the first embodiment.

The emulsion of the first embodiment is an oil-in-water emulsion. Particle sizes (expressed as Z-average) are typically in the range of from 20 nm to 400 nm, preferably 50 nm to 300 nm and most preferably 80 nm to 200 nm. The polydispersity index (PDI) is typically in the range of from 0.05 to 0.40, preferably 0.08 to 0.30 and most preferably 0.10 to 0.20.

The emulsion of the first embodiment is advantageous because excellent storage stability is typically observed. For instance, in certain aspects of the first embodiment, the emulsion shows a variation of less than 15%, preferably less than 10% and more preferably less than 5%, in Z-average particle size when being stored at 5° C. for 3 months in a glass container, protected from light. In a related aspect of the first embodiment, the emulsion shows a variation of less than 15%, preferably less than 10% and more preferably less than 5%, in Z-average particle size when being stored at 22° C. for 3 months in a glass container, protected from light. In further specific aspects, the emulsion shows a variation of less than 30%, preferably less than 25% and more preferably less than 20%, in polydispersity index when being stored at 5° C. for 3 months in a glass container, protected from light. In yet another specific aspect, the emulsion shows a variation of less than 30%, preferably less than 25% and more preferably less than 20%, in polydispersity index when being stored at 22° C. for 3 months in a glass container, protected from light. In all these aspects, the variation is calculated using the following formula:

$$variation [ \% ] = 100 \% * \vert initial value - value at 3 months \vert / initial value .$$

There are, however, some less preferred aspects of the emulsion of the first embodiment, which merely exhibit short-term stability, for instance stability in accordance with the above criteria, but only for 1 week, or 1 day or 4 hours or even only 2 hours, 1 hour or even 30 minutes. Depending on the envisaged use of the emulsion, such short periods of stability may be sufficient. The emulsions of these aspects of the present invention still benefit from the advantage of being suitable for emulsification by a low energy method or, more preferably, an ultra-low energy method such as shaking by hand (also referred to as manual shaking).

### Composition for Forming the Emulsion of the Invention

The composition for forming the emulsion of the first embodiment of the invention is characterized by the same components as specified hereinabove for the emulsion of the first embodiment of the invention. These are present in the same relative amounts as specified hereinabove for the emulsion of the first embodiment of the invention.

However, contrary to the emulsion of the first embodiment of the invention, the composition for forming the emulsion of the invention is not in the form of an emulsion.

According to a preferred aspect, the composition for forming the emulsion of the invention is provided in a closed container that is suitable for forming the emulsion of the first embodiment of the invention by shaking by hand. It is further preferred to provide the composition for forming the emulsion of the invention in the closed container together with instructions for preparing an emulsion by means of shaking by hand. Such instructions preferably indicate that shaking should be carried out for a time period of at least 1 s, more preferably 1.5 s to 120 s, even more preferably 1.5 to 60 s and most preferably 2 s to 30 s.

### Premix

The premix of the first embodiment of the invention is characterized by the presence of the components, as specified above for the emulsion of the first embodiment of the invention, except for the absence of the water component. However, indications of relative amounts must be adjusted to take the absence of the water component into account, wherein adjustment means that ratio of ester component to surfactant and ester component plus surfactant to optional further component should remain as specified above such that simple dilution with aqueous component yields a composition that is in agreement with the relative amount indications specified above.

For example, for a given emulsion of interest, the relative amounts of ester component, surfactant and water component may be e %, s % and w %, respectively, with e %+s %+w %=100%. The corresponding premix is only formed by ester component and surfactant with relative amounts of e1% and s1%, respectively, with e1%+s1%=100% of the premix. 100%−w % of the emulsion correspond to 100% of the premix. This means that the individual percentages must be scaled by a factor of f=[100%/(100%−w %)]. Hence, e1% and s1% can be calculated based on the known relative amounts e %, s % and w %, respectively, using the following equations e1%=f*e % and s1%=f*s %, respectively.

If additionally an optional component is present in the emulsion of interest in a relative amount of 0%, the above approach can be applied in an analogous manner by relying on modified equations e %+s %+w %+o %=100% of emulsion; e1%+s1%+o1%=100% of premix; f=[100%/(100%−w %)]; e %=f*e %; s1%=f*s %; and o1%=f*o %.

### Methods of Preparation

The emulsion of the first embodiment of the invention can be prepared with any low energy method or ultra-low energy method, wherein energy input is as defined above for these methods.

For instance, suitable low energy methods include mechanical stirring, automatic shaking, low energy sonication.

Preferably, an ultra-low energy method is used. Suitable ultra-low energy methods include shaking by hand, manual stirring, pouring, spinning, straining, and blending. According to the first embodiment of the invention, it is particularly preferred to perform emulsification by shaking by hand.

Manual shaking is preferably carried out for a time period of at least 1 s, more preferably 1.5 s to 120 s, even more preferably 1.5 to 60 s and most preferably 2 s to 30 s. The frequency of shaking is preferably 1-6 Hz and more preferably 2-4 Hz. The preferred manual shaking includes 4 to 20 shakes or more and more preferably 10 to 120 shakes.

### Uses of the Emulsion of the First Embodiment

There is no particular restriction to the possible uses of the emulsion of the first embodiment. This emulsion is suitable for any practical application, in which emulsions of comparable particle sizes are used. This includes, in particular, the administration of contrast agents for CT or PET-CT imaging such as visualization of liver conditions or brown and/or beige adipose tissue or detection of cachexia or precachexia, as a described in detail in WO 2019/030024 and in WO 2020/165349, respectively.

Another area of applications of the emulsion of the first embodiment is the field of cosmetics. Again, it is a requirement that the active agents in the desired relative amounts do not interfere with the formation of the emulsion of the first embodiment (which is to be judged based on the test specified above in connection with definition 01).

Further conceivable uses of the emulsion of the first embodiment include food industrial applications, personal hygiene products, emulsified fuels, polymer chemical synthesis (glues, paints and synthetic latexes).

### Second Embodiment

Components to be Used The ester component can be selected from the ester components according to the above definition E2 or E4. For the remaining components, there is no particular restriction in the context of the second embodiment. A water component can be a used according to definition W1 or W2. A surfactant can be used according to definition S1, S2 or S3. The indications of preference provided above apply to the components to be used in the second embodiment.

In addition to the above-mentioned essential components, it is possible to use one or more optional further components in accordance with definition 01 or to omit such optional further components in accordance with definition 02. In preferred aspects of the second embodiment, at least one such further component is present.

### Relative Amounts of Components

The components of the emulsion, composition and premix of the second embodiment may be present in the same relative amounts as specified above for the first embodiment.

### Methods of Preparation

The emulsion of the second embodiment may be prepared from the composition of the second embodiment using any emulsification method. This includes high energy emulsification methods using devices such as high-shear mixers, microfluidizers, high-pressure homogenizers, jet disperser or ultrasonication devices; and low energy or ultra-low energy emulsification methods as described for the first embodiment above. Reference is furthermore made to the emulsification methods described in WO 2019/030024 A and WO 2020/165349 A.

Low energy or ultra-low energy emulsification methods as described for the first embodiment above are preferably used. Ultra-low energy emulsification methods as described for the first embodiment above are particularly preferable.

### Emulsion

The emulsion of the second embodiment is an oil-in-water emulsion and it is characterized by the same properties and especially by the same particle sizes and/or polydispersity indexes and/or storage stabilities as specified above for the emulsion of the first embodiment.

### Composition for Forming the Emulsion of the Invention

The above indications for the composition for forming the emulsion of the first embodiment apply also to the composition for forming the emulsion of the second embodiment. That is, the composition for forming the emulsion of the second embodiment of the invention is characterized by the same components as specified hereinabove for the emulsion of the second embodiment of the invention, which means that, contrary to the first embodiment, no iodine-containing fatty acid-derived moiety in the ester component is present. The components of the second embodiment are present in the same relative amounts as specified hereinabove for the emulsion of the second embodiment of the invention.

However, contrary to the emulsion of the second embodiment of the invention, the composition for forming the emulsion of the invention is not in the form of an emulsion.

According to a preferred aspect, the composition for forming the emulsion of the second embodiment of the invention is provided in a closed container that is suitable for forming the emulsion of the second embodiment of the invention, for instance by shaking by hand. It is further preferred to provide the composition for forming the emulsion of the invention in the closed container together with instructions for preparing an emulsion by means of shaking by hand. Such instructions preferably indicate that shaking should be carried out for a time period of at least 1 s, more preferably 1.5 s to 120 s, even more preferably 1.5 s to 60 s and even more preferably 2 s to 30 s. In addition, or alternatively, the instructions may also refer to other methods of emulsification as specified above for the second embodiment, such as sonication.

### Premix

The premix of the second embodiment of the invention corresponds essentially to the premix of the first embodiment, with the exception that no iodine-containing ester component is present. Specifically, the premix of the second embodiment of the invention is characterized by the presence of the components, as specified above for the emulsion of the second embodiment of the invention, except for the absence of the water component. Indications of relative amounts must be adjusted to take the absence of the water component into account, wherein adjustment means that the ratios of ester component to surfactant and ester component plus surfactant to optional further component should remain as specified above such that simple dilution with aqueous component yields a composition that is in agreement with the relative amount indications specified above.

The approach for calculating relative amounts of the components in the premix, as described above in relation to the first embodiment, is applicable also for the premix of the second embodiment.

### Uses of the Emulsion of the Second Embodiment

The emulsion of the second embodiment can be used for the same purposes and applications as described above with respect to the first embodiment. However, the CT or PET-CT imaging applications based on an iodine-containing ester component of definition E1 are not generally possible since only ester components in accordance with definition E2 or E4 are used in the second embodiment, in which the R₁ group contains no iodine substituent. Hence, the CT or PET-CT imaging applications are possible only if an ester component is used, in which the R₂ group carries one or more iodine substituents, preferably at least three iodine substituents per molecule.

### Third Embodiment

### Ester Component

In the third embodiment of the invention, it is possible to use the ester component according to definition E1 above. Preferably, the ester component is in accordance with definition E2 and more preferably it is according to definition E3 or definition E4.

### Aqueous Phase

In the third embodiment, it is possible to use a water component according to the above definitions W1 or W2. Preferably, the water component is in accordance with definition W2.

### Surfactant

In the third embodiment, it is possible to use a surfactant according to the above definitions S1, S2 or S3. Preferably, the surfactant component is in accordance with definition S3.

Further Optional Components The emulsion, composition and premix of the third embodiment may also contain one or more optional further components in accordance with definition O1 above. However, in some aspects of the third embodiment, optional further components may be absent in accordance with definition O2 above.

### Combinations of Components

In one aspect of the third embodiment of the invention, the ester component is selected such that it is in accordance with definition E3 above, while there are no particular restrictions regarding the other components. That is, the water component may be selected from definition W1 or preferably definition W2, and the surfactant may be selected from definition S1, preferably definition S2, or even more preferably definition S3. Optional further components may or may not be present in accordance with definitions O1, or O2.

In another aspect of the third embodiment of the invention, the water component is selected from definition W2, while there are no particular restrictions regarding the other components. That is, the ester component is selected such that it is in accordance with any of the definitions E1, preferably E2 or more preferably E3 or E4 above and the surfactant may be selected from definition S1 or preferably definition S2 or more preferably definition S3. Optional further components may or may not be present in accordance with definitions O1, or O2.

In yet another aspect of the third embodiment of the invention, the surfactant component is selected from definition S3, while there are no particular restrictions regarding the other components. That is, the ester component is selected such that it is in accordance with any of the definitions E1, preferably E2 or more preferably E3 or E4 above and the water component may be selected from definition W1 or preferably definition W2. Optional further components may or may not be present in accordance with definitions O1, or O2.

### Relative Amounts of Components

The components of the third embodiment may be present in the emulsion of the third embodiment in the same relative amounts as specified above for the first embodiment.

### Methods of Preparation

In the third embodiment, there is no particular restriction regarding the methods that can be used for preparing the emulsion. Any of the methods listed for the second embodiment can be used. It is however preferred to employ a low-energy or ultra-low-energy method as described above for the first embodiment and it is particularly preferred to employ shaking by hand.

### Emulsion

The emulsion of the third embodiment is characterized by the presence of the components specified above for the third embodiment in the above-specified relative amounts.

Apart from that, the emulsion of the third embodiment is characterized by the same properties as described above for the first embodiment. It is an oil-in-water emulsion and it typically shows the Z-average particle size and/or polydispersity index and/or storage stability as specified above for the first embodiment.

### Composition for Forming the Emulsion of the Invention

The composition for forming the emulsion of the third embodiment of the invention is characterized by the same components as specified hereinabove for the emulsion of the third embodiment of the invention. These are present in the same relative amounts as specified hereinabove for the emulsion of the third embodiment of the invention.

However, contrary to the emulsion of the third embodiment of the invention, the composition for forming the emulsion of the invention is not in the form of an emulsion.

According to a preferred aspect, the composition for forming the emulsion of the third embodiment of the invention is provided in a closed container that is suitable for forming the emulsion of the third embodiment of the invention by shaking by hand. It is further preferred to provide the composition for forming the emulsion of the invention in the closed container together with instructions for preparing an emulsion by means of shaking by hand. Such instructions preferably indicate that shaking should be carried out for a time period of at least 1 s, more preferably 1.5 s to 120 s, even more preferably 1.5 s to 60 s and even more preferably 2 s to 30 s. In addition or alternatively, the instructions may also refer to other methods of emulsification as specified above for the second embodiment, such as sonication.

### Premix

The premix of the third embodiment of the invention can be combined with the water component described above for the third embodiment to thereby form the above composition for forming the emulsion of the third embodiment of the invention.

The premix of the third embodiment corresponds essentially to the premixes of the first and second embodiment, but with the condition that the ester component, surfactant and any optional further component must be in accordance with the above description of the components of the emulsion of the third embodiment (except for absence of the water component).

Indications of relative amounts must be adjusted to take the absence of the water component into account, wherein adjustment means that the ratios of ester component to surfactant and ester component plus surfactant to optional further component should remain as specified above such that simple dilution with aqueous component yields a composition that is in agreement with the relative amount indications specified above.

The approach for calculating relative amounts of the components in the premix, as described above in relation to the first embodiment, is applicable also for the premix of the third embodiment.

### Uses of the Emulsion of the Third Embodiment

The emulsion of the third embodiment can be used for the same purposes and applications as described above with respect to the first and second embodiment.

### Preferred Embodiments

As indicated earlier, the present invention relates to three main embodiments. For each of these embodiments, multiple different aspects and ways of implementation are described. These descriptions also include indications of preference. It is even more preferred to implement the present invention by combining such preferred aspects for two or more features unless such combinations or preferred features are not feasible or not meaningful from a scientific or practical point of view. For example, it is preferred to use a combination of a preferred ester component with a preferred surfactant component and/or a preferred water component. It is even more preferred to use these components in relative amounts that are indicated as being preferred. However, if two such preferred components should be mutually incompatible, for instance due to degradation problems or formation of insoluble complexes, such a combination of preferred features should be avoided and is consequently not preferred.

Preferred embodiment 1-1: In particular, one such preferred aspect of the first embodiment, as specified in the above description and the below claims, relates to an emulsion, a product for emulsification, a premix product and a method for producing an emulsion as described hereinabove and below for the first embodiment, but which are based on combination of the following preferred features:
an ester component of formula I, wherein
R₁ is selected from the group consisting of linear-chain alkyl groups, branched-chain alkyl groups, linear-chain alkenyl groups, branched-chain alkenyl groups, linear-chain alkynyl groups, branched-chain alkynyl groups, each of these optionally contain one cyclopropyl group or cyclobutyl group and each of these groups having a total of 13 to 19 carbon atoms and 1 to 4 substituents, the substituents being selected from C₁₋₄-alkoxy, C₁₋₄-thioether, CN, N₃, fluorine, chlorine, bromine and iodine;
R₂ is selected from a linear C₁₋₄ alkyl group, a branched C₃₋₄ alkyl group, a linear C₂₋₄ alkenyl group, a branched C₃₋₄ alkenyl group, a linear C₂₋₄ alkynyl group and a branched C₄ alkynyl group, each of these groups being unsubstituted or containing 1 to 4 substituents selected from fluorine, chlorine, bromine, iodine, linear C₁₋₆ alkyl, branched C₃₋₆ alkyl, cyclic C₃₋₆ alkyl, linear C₂₋₆ alkenyl, branched C₃₋₆ alkenyl, cyclic C₅₋₆ alkenyl, linear C₂₋₆ alkynyl, branched C₄₋₆ alkynyl, C₅₋₁₄ aryl, 5-14-membered heteroaryl, cyano, thiol, thioether, nitro, azido, and hydroxyl, with the proviso that if each of the substituent and the substituted group is a saturated or unsaturated aliphatic group (which may include a saturated or unsaturated cyclic group but not a cyclic aromatic group), instead of the individual carbon atom restrictions, the total number of carbon atoms of the substituted group must be in accordance with the carbon atom range resulting from an addition of the permitted carbon atom numbers for the substituent and the substituted group;
the surfactant is selected from polyoxyethylene castor oils having a total number of 4 to 200, preferably 5 to 60, oxyethylene units, and polyoxyethylene sorbitan fatty acid esters wherein the total number of oxyethylene units is 4 to 20 and the fatty acid is a fatty acid having 12 (e.g. lauric acid) to 18 (e.g. stearic or oleic acid) carbon atoms, which may be saturated or unsaturated, wherein the polyoxyethylene castor oil or polyoxyethylene sorbitan fatty acid ester is more preferably selected from the specific surfactants listed in the corresponding groups (S11a) and (S12a) above;
the water component is not particularly restricted and a further optional component is optionally present; wherein
the ester component-to-surfactant ratio is between 75:25 and 40:60 (w/w), and, in the aspects other than the premix: the water-to-surfactant ratio is between 400:1 and 65:35 and the water-to-ester component ratio is between 400:1 and 45:55 (w/w); and
wherein the emulsification is to be carried out by an ultra-low energy method such as shaking by hand.

Preferred embodiment 1-2: An even more preferred embodiment is characterized by the same features as the preferred embodiment 1-1 above, wherein R₁ group is a linear C₁₆-C₁₈ alkyl group or a linear C₁₆-C₁₈ alkenyl group, each of which may optionally be substituted with 1, 2 or 3 halogen atoms selected from F, Cl and Br.

Preferred embodiment 1-3: An even more preferred embodiment is characterized by the same features as the preferred embodiment 1-1 or 1-2 above, with the exception that R₂ is selected from C₁₋₄-alkyl groups such as methyl, ethyl, propyl, isopropyl, butyl, isobutyl, and t-butyl.

Preferred embodiment 1-4: An even more preferred embodiment is characterized by the same features as the preferred embodiment 1-1, 1-2 or 1-3 above, wherein the ester component-to-surfactant ratio is between 10:1 and 70:30 (w/w), preferably between 50:50 and 70:30 (w/w), and, in the aspects other than the premix: the water-to-surfactant ratio is between 100:1 and 80:20 (w/w) and the water-to-ester component ratio is between 100:1 and 1:1 (w/w).

Preferred embodiment 1-5: An even more preferred embodiment is characterized by the same features as the preferred embodiment 1-1, 1-2, 1-3 or 1-4 above, wherein one or more further components is/are present in a relative amount of 0 to 5 wt % based on the total weight of the emulsion (or product for emulsification). Of course, in the case of the premix, the further component is present in a greater relative amount (due to the absence of water), such as from more than 0 wt. % to 10 wt. %. In case of multiple further components, each individual optional component is present in an amount of from more than 0 to 1 wt. %, more preferably from more than 0 to 0.5 wt. % based on the total weight of the emulsion (or product for emulsification) and from more than 0 to 2 wt. %, more preferably from more than 0 to 1 wt. % based on the total weight of the premix, as the case may be.

Preferred embodiment 1-6: An even more preferred embodiment is characterized by the same features as the preferred embodiment 1-1, 1-2, 1-3, 1-4 or 1-5 above, wherein the water component is selected from drinking water such as tap water, mineral water, spring water, or carbonated water, coffee, tea, milk, fruit juice, and soft drinks, and more preferably it is selected from tap water, spring water, mineral water, coffee, and tea.

Preferred embodiment 1-7: A further preferred embodiment is characterized by the same features as any one of the preferred embodiments 1-1 to 1-6, except that the ester component-to-surfactant ratio is between 20:1 and 45:55 (w/w), and, in the aspects other than the premix: the water-to-surfactant ratio is between 400:1 and 65:35 and the water-to-ester component ratio is between 400:1 and 45:55 (w/w).

Preferred embodiment 1-8: A further preferred embodiment is characterized by the same features as any one of the preferred embodiments 1-1 to 1-7, except that the ester component-to-surfactant ratio is between 20:1 and 45:55 (w/w), and, in the aspects other than the premix: the water-to-surfactant ratio is between 400:1 and 65:35 and the water-to-ester component ratio is between 400:1 and 50:50 (w/w).

Preferred embodiment 1-9: A further preferred embodiment is characterized by the same features as any one of the preferred embodiment 1-1 to 1-3 except that the surfactant is selected from poloxamers. For this embodiment, the ester component-to-surfactant ratio is between 10:1 and 45:55 (w/w), and, in the aspects other than the premix: the water-to-surfactant ratio is between 95:5 and 50:50, preferably between 90:10 and 70:35, and the water-to-ester component ratio is between 80:20 and 50:50 (w/w).

Preferred embodiment 1-10: A further preferred embodiment is characterized by the same features as any one of the preferred embodiment 1-1 to 1-3 except that the surfactant is selected from monoglycerides of fatty acids. For this embodiment, the ester component-to-surfactant ratio is between 10:1 and 70:35 (w/w), and, in the aspects other than the premix: the water-to-surfactant ratio is between 98:2 and 70:35 and the water-to-ester component ratio is between 25:1 and 50:50 (w/w).

Preferred embodiment 2-1: A preferred aspect of the second embodiment, as specified in the above description and the below claims, relates to an emulsion, a product for emulsification, a premix product and a method for producing an emulsion as described hereinabove and below for the second embodiment, but is based on a combination of the preferred features as specified for anyone of preferred embodiments 1-1 to 1-10 above with the sole exception that the R₁ group of the ester component does not carry any iodine substituent.

Preferred embodiment 2-2: An even more preferred embodiment is as characterized by the same features as the preferred embodiment 2-1 above, but with the additional condition that neither R₁ nor R₂ of the ester component carries any iodine substituent.

Preferred embodiment 3-1: A preferred aspect of the third embodiment, as specified in the above description and the below claims, relates to an emulsion, a product for emulsification, a premix product and a method for producing an emulsion as described hereinabove and below for the third embodiment, but is based on a combination of the preferred features as specified for anyone of preferred embodiments 1-1 to 1-10 and/or wherein the ester component is in accordance with the above definition E3 or definition E4, the water component is in accordance with definition W2 and the surfactant is in accordance with definition S1, S2 or S3.

Preferred embodiment 3-2: An even more preferred embodiment is as characterized by the same features as the preferred embodiment 3-1 above, but wherein the surfactant component is in accordance with definition S3.

### EXAMPLES

Particle size was measured using dynamic light scattering using a 173° angle on a Zetasizer Nano ZS (Malvern Panalytical Ltd, United Kingdom) using a 4 mW He—Ne Laser (633 nm) in polystyrene micro cuvettes from Brand (Wertheim, Germany). Samples were diluted 1:100 in milliQ water to reach an attenuator index between 4 and 6. Three measurements were performed for each sample at 25° C. Z-average and polydispersity index were recorded as average±standard deviation of the three measurements. The size distribution and mean diameter were calculated from the data using ZetaSizer software v8.

Unless otherwise stated, the amount ratios of the components, such as water-to-oil ratio and oil-to-surfactant ratio, refer to weight ratios.

### Example 1: Ethyl Oleate Nanoemulsions

Ethyl oleate and a single polysorbate (Tween 20, Tween 40, Tween 60 or Tween 80) were mixed in a 2:1 ratio. Water was added (water-to-oil ratio 1.8:1) and the mixture shaken by hand for 20 seconds. Nanoemulsions were obtained, with particle size below 200 nm and PDI below 0.2 (Table 1).

TABLE 1
Particle size for ethyl oleate nanoemulsions

| Surfactant | Tween 20 | Tween 40 | Tween 60 | Tween 80 |
|---|---|---|---|---|
| Z-average (nm) | 164.6 ± 1.7 | 168.2 ± 0.4 | 150.5 ± 0.6 | 144.7 ± 1.0 |
| PDI | 0.144 ± 0.008 | 0.162 ± 0.004 | 0.109 ± 0.007 | 0.121 ± 0.02 |

### Example 2: Ethyl Esters of Poppy Seed Oil Nanoemulsions

Ethyl esters of poppy seed oil and a single surfactant (Tween 80, or Etocas 35, or Kolliphor RH40) were mixed in a 2:1 ratio. Water was added (water-to-oil ratio 1.8:1) and the mixture shaken for 20 seconds. Nanoemulsions were obtained, with particle size below 200 nm and PDI 0.2 or below.

TABLE 2
Particle size for ethyl esters of poppy seed oil nanoemulsions

| Surfactant | Tween 80 | Etocas 35 | Kolliphor RH40 |
|---|---|---|---|
| Z-average (nm) | 169.5 ± 0.2 | 169.2 ± 1.6 | 191.6 ± 0.9 |
| PDI | 0.200 ± 0.01 | 0.147 ± 0.009 | 0.184 ± 0.010 |

### Example 3: Ethyl Oleate and Tween 60 Nanoemulsion

Ethyl oleate and Tween 60 were mixed in various proportions. Water was added and the mixture shaken for 10 seconds. The final volume was 1 mL or less. A pseudo-ternary diagram was created. The stable nanoemulsion domain was found to be between 4:1 and 1:2 (ester component-to-surfactant ratio), between 9:1 and 45:55 (water-to-ester component ratio), and between 98:2 and 4:3 (water-to-surfactant ratio). The proportion of ester component that yields a nanoemulsion is between 5 and 45% (FIG. 1).

### Example 4: Ethyl Esters of Poppy Seed Oil and Tween 80 Nanoemulsion

Ethyl esters of poppy seed oil and Tween 80 were mixed in various proportions. Water was added and the mixture shaken for 10 seconds. The final volume was 1 mL or less. A pseudo-ternary diagram was created. The nanoemulsion domain was found to be between 9:1 and 1:1 (ester component-to-surfactant ratio), between 85:15 and 45:55 (water-to-ester component ratio) and between 95:5 and 3:2 (water-to-surfactant ratio); the proportion of ester component that yields a nanoemulsion is between 12 and 45% (FIG. 2).

### Example 5: Nanoemulsion Made of Ethyl Di-Iodo Stearate and Kolliphor RH40

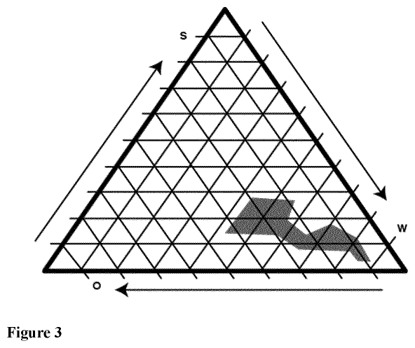

Ethyl di-iodo stearate and Kolliphor RH40 were mixed in various proportions. Water was added and the mixture shaken for 5 seconds. The final volume was 1 mL or less. A pseudo-ternary diagram was created. The nanoemulsion domain was found to be between 9:1 and 35:65 (ester component-to-surfactant ratio), between 9:1 and 1:1, (water-to-ester component ratio) and between 95:5 and 3:2 (water-to-surfactant ratio); the proportion of ester component in the final product that yields a nanoemulsion is between 8 and 45% (FIG. 3).

### Example 6: Scale Up of Method of Preparation

Ethyl oleate and Tween 60 were mixed at room temperature in a 2:1 ratio. Water (280 mL) was added to obtain a final mass of 0.5 kg. The mixture was shaken for 60 seconds by hand. The nanoemulsion displayed a homogeneous appearance. The particle size and PDI were 143.1±1.4 nm and 0.153+0.028, respectively. This is in accordance with results obtained with smaller volumes and smaller scale. The shake method is easily scalable to multi-kilogram scale.

### Example 7: Isopropyl Myristate Nanoemulsions

Isopropyl myristate and a single surfactant (Tween 80 or Kolliphor RH40) were mixed in a 2:1 ratio. Water was added and the mixture shaken for 20 seconds. Nanoemulsion was obtained, with particle size (Z-average) below 300 nm and PDI below 0.3 (Table 3).

TABLE 3
Particle size for isopropyl myristate nanoemulsions

| Surfactant | Tween 80 | Kolliphor RH40 |
|---|---|---|
| Z-average (nm) | 221.3 ± 1.4 | 141.8 ± 0.9 |
| PDI | 0.239 ± 0.008 | 0.199 ± 0.004 |

### Example 8: Comparison Between Different Emulsification Processes

Different formulations made of ethyl iodostearate (30% w/w), Kolliphor RH 40 (12% w/w) and water (ad 1 g) were prepared using different emulsification methods:
Nanoprecipitation: The oil and surfactant were dissolved in 1 mL acetone. This mixture was added slowly (one drop/s) to water under magnetic stirring. After complete addition, the acetone was removed under reduced pressure at 40° C.
Sonication: The oil and surfactant were mixed with water. This mixture was sonicated for 15 min in an ultrasound bath.
Phase inversion—stepwise addition: The oil and surfactant were mixed using a magnetic stirrer. The mixture was heated at 60° C. The water was heated at 60° C. The water was added dropwise (1 drop/s) to the oil phase under stirring. Stirring was pursued until the mixture was back to room temperature.
Phase inversion—direct addition: The oil and surfactant were mixed using a magnetic stirrer. The mixture was heated at 60° C. The water was heated at 60° C. The water was added to the oil-surfactant mixture under stirring. Stirring was pursued until the mixture was back to room temperature.
Classical emulsification—stepwise addition: The oil and surfactant were mixed using a magnetic stirrer. The mixture was heated at 60° C. The water was heated at 60° C. The oil phase was added to the water dropwise (1 drop/s) under stirring. Stirring was pursued until the mixture was back to room temperature.
Classical emulsification—direct addition: The oil and surfactant were mixed using a magnetic stirrer. The mixture was heated at 60° C. The water was heated at 60° C. The oil phase was added to the water under stirring. Stirring was pursued until the mixture was back to room temperature.

The appearance, particle size, total preparation time (including weighing, preparation of equipment, heating and cooling down time (if any), etc.) and required equipment were recorded in Table 4. The nanoprecipitation method yielded no nanoemulsion. The other methods resulted in the formation of nanoemulsions with particle sizes ranging from 160 to 273 nm and PDI ranging from 0.09 to 0.292. In this regard, the nanoemulsion with the largest particles and distribution was the phase inversion with stepwise addition of water. All other methods resulted in nanoemulsions with similar particle size and PDJ. Preparation time ranged from 20 minutes (sonication and “shake” methods) to 40 minutes. The nanoprecipitation was the most time-consuming method (1 hour) but resulted in no nanoemulsion. The shaking method requires the least equipment and is very efficient.

TABLE 4
Results from the same composition made using different emulsification methods

| Homogenization method | Required equipment | Preparation time | | Z-average (nm) | PDI |
|---|---|---|---|---|---|
| Nanoprecipitation | Rotavapor | 1 | hour | N/A | N/A |
| Sonication (ultrasound bath) | Sonicator | 20 | minutes | 184.8 ± 0.7 | 0.204 ± 0.016 |
| Phase inversion (direct) | Heating pad 60° C. | 40 | minutes | 160.1 ± 0.3 | 0.086 ± 0.009 |
| Phase inversion (stepwise) | Heating pad 60° C. | 40 | minutes | 273.3 ± 11.4 | 0.292 ± 0.04 |
| Classical emulsification (direct) | Heating pad 60° C. | 40 | minutes | 149.8 ± 0.6 | 0.118 ± 0.006 |
| Classical emulsification (stepwise) | Heating pad 60° C. | 40 | minutes | 164.2 ± 0.7 | 0.134 ± 0.016 |
| Claimed method | None | 5 | minutes | 165.0 ± 0.8 | 0.135 ± 0.006 |

### Example 9: Modification of Water Phase

Ethyl esters of poppy seed oil and Tween 80 were mixed in a 4:1 ratio. Different water phases (purified water, sparkling water (San Pellegrino), cold coffee (l'Or Espresso Splendid) with milk, fruit juice (multifruit, no preservatives), lukewarm tea (Twinings Earl grey)) were added (water-to-oil ratio 4:1) to reach 1.4 g of final formulation. The formulations were shaken for 5 seconds by hand. Appearance and particle size were assessed. All formulations were homogeneous and milky, with color depending on the water phase used (white for purified and sparkling water, light brown for tea and coffee, light orange for fruit juice). All formulations displayed similar particle size (Z-average: between 142.5 and 153.4 nm; PDJ: between 0.102 and 0.149) (Table 5).

TABLE 5
Particle size of formulations prepared with different
water phases. Results presented as average ± STD

| Water phase | Z-average (nm) | PDI |
|---|---|---|
| Purified water | 144.1 ± 0.5 | 0.117 ± 0.005 |
| Saline (0.9% NaCl) | 145.2 ± 1 | 0.141 ± 0.010 |
| Sparkling water - San Pellegrino ® | 142.5 ± 0.9 | 0.102 ± 0.011 |
| Coffee with milk | 146.3 ± 0.9 | 0.149 ± 0.005 |
| Fruit juice | 148.7 ± 1.6 | 0.140 ± 0.011 |
| Tea | 153.4 ± 2.1 | 0.145 ± 0.014 |

### Example 10: Stability Study

Ethyl di-iodo stearate and Etocas 35 were mixed in a 1.75:1 ratio. Water was added (water-to-oil ratio 1.3:1) and the mixture was shaken by hand for 10 seconds. The resulting emulsions were kept at room temperature (RT) and 2-8° C. The particle size and appearance were assessed after 1 day, 2 weeks, 1, 2 and 3 months. The appearance of the formulation remained unchanged during the stability study. Particle size did not significantly change, as presented in Table 6.

TABLE 6
Particle size of ethyl di-iodo stearate + Etocas 35 emulsion at room
temperature and 2-8° C. after 2 weeks, 1, 2 and 3 months

| | | 1 day | 2 weeks | 1 month | 2 months | 3 months |
|---|---|---|---|---|---|---|
| Z-average | 5° C. | 92.7 ± 1.0 | 97.4 ± 1.2 | 98.2 ± 0.2 | 97.2 ± 0.4 | 98.4 ± 0.6 |
| (nm) | RT | 106 ± 0.2 | 101.6 ± 0.5 | 103.4 ± 0.4 | 105.8 ± 0.2 | 104.7 ± 0.7 |
| Polydispersity | 5° C. | 0.113 ± 0.011 | 0.115 ± 0.012 | 0.130 ± 0.01 | 0.114 ± 0.005 | 0.133 ± 0.003 |
| index | RT | 0.136 ± 0.013 | 0.125 ± 0.014 | 0.116 ± 0.008 | 0.128 ± 0.004 | 0.112 ± 0.022 |

### Example 11: Influence of Shaking Time

Ethyl di-iodo stearate and Kolliphor RH40 were mixed in a 2.5:1 ratio. Water was added (water-to-oil ratio 2:1) to reach 350 mg. The duration of shaking was varied between samples. The particle size was then analysed by DLS. Shaking for more than 1 second or 3 shakes or more is required to obtain a nanoemulsion with particle size below 200 nm and PDI below 0.2 (Table 7).

TABLE 7
Influence of shake duration on particle size and polydispersity index

| | | | | | | | |
|---|---|---|---|---|---|---|---|
| Shake | 1 | 1.5 | 2 | 5 | 10 | 20 | 30 |
| length (s) | | | | | | | |
| Number | 2 | 3 | 4 | 10 | 20 | 40 | 60 |
| of shakes | | | | | | | |
| Z-average | 222.2 ± 5.8 | 158.7 ± 1.3 | 138.1 ± 1 | 133.8 ± 1 | 153.7 ± 0.6 | 174.8 ± 1.6 | 127.2 ± 0.7 |
| (nm) | | | | | | | |
| PDI | 0.515 ± 0.1 | 0.106 ± 0.027 | 0.109 ± 0.017 | 0.087 ± 0.009 | 0.097 ± 0.017 | 0.060 ± 0.019 | 0.058 ± 0.010 |

### Example 12: Butyl Stearate Nanoemulsions

Butyl stearate heated at 30° C. (above melting point) and a surfactant (Tween 40, Tween 80 or Kolliphor RI40) were mixed in a 2:1 ratio. Water was added and the mixture shaken for 20 seconds. Nanoemulsions were obtained, with particle size (Z-average) below 200 nm and PDI below 0.4 (Table 8).

TABLE 8
Particle size for butyl stearate nanoemulsions

| Surfactant | Tween 40 | Tween 80 | Kolliphor RH40 |
|---|---|---|---|
| Z-average (nm) | 118 ± 1.9 | 112.9 ± 1.4 | 120.5 ± 0.3 |
| PDI | 0.377 ± 0.006 | 0.229 ± 0.010 | 0.286 ± 0.028 |

### Example 13: Methyl Linoleate Nanoemulsions

Methyl linoleate and a surfactant (Tween 40, Tween 80 or Kolliphor RH40) were mixed in a 2:1 ratio. Water was added and the mixture shaken for 20 seconds. Nanoemulsions were obtained, with particle size (Z-average) below 300 nm and PDI below 0.4 (Table 9).

TABLE 9
Particle size for methyl linoleate nanoemulsions

| Surfactant | Tween 40 | Tween 80 | Kolliphor RH40 |
|---|---|---|---|
| Z-average (nm) | 261.1 ± 4.9 | 263.2 ± 1.4 | 232.1 ± 2 |
| PDI | 0.193 ± 0.041 | 0.208 ± 0.020 | 0.333 ± 0.022 |

### REFERENCES

1. Aswathanarayan, J. B. & Vittal, R. R. Nanoemulsions and Their Potential Applications in Food Industry. Front. Sustain. Food Syst. 3, (2019).
2. Anton, N. & Vandamme, T. F. Nano-emulsions and micro-emulsions: clarifications of the critical differences. Pharm. Res. 28, 978-985 (2011).
3. Anton, N. & Vandamme, T. F. The universality of low-energy nano-emulsification. Int. J. Pharm. 377, 142-147 (2009).
4. Buya, A. B., Beloqui, A., Memvanga, P. B. & Préat, V. Self-Nano-Emulsifying Drug-Delivery Systems: From the Development to the Current Applications and Challenges in Oral Drug Delivery. Pharmaceutics 12, (2020).
5. Miyagawa, Y., Shima, M., Matsuno, R. & Adachi, S. Energy Efficiency of Different Emulsification Methods: A Comparative Evaluation. Jpn. J. FoodEng. 16, 71-74 (2015).

1. An emulsion which is selected from the following embodiments (Ae), (Be), (Ce1), (Ce2), (Ce3) and (Ce4),
(Ae) an emulsion consisting of an ester component, a surfactant, a water component and one or more optional further components, wherein
the ester component is one or more compounds each characterized by the general Formula I: 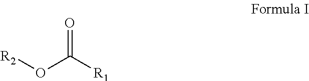
wherein R₁ represents a hydrocarbon group, which contains 30 or less carbon atoms and which is optionally substituted by 1 to 6 substituents each independently selected from fluorine, chlorine, bromine and iodine,
R₂ represents an optionally substituted group selected from the group consisting of linear-chain C₁₋₁₂ alkyl groups, branched-chain C₃₋₁₂ alkyl groups, cyclic C₃₋₁₂ alkyl groups, linear-chain C₂₋₁₂ alkenyl groups, branched-chain C₃₋₁₂ alkenyl groups, cyclic C₅₋₁₂ alkenyl groups, linear-chain C₂₋₁₂ alkynyl groups, branched-chain C₄₋₁₂ alkynyl groups, C₅₋₁₄ aryl groups, 5-14-membered heteroaryl groups and 5-14 membered saturated or partly unsaturated non-aromatic heterocycles with 1 to 4 heteroatoms independently selected from N, O and S;
the surfactant is one or more non-ionic surfactant compounds;
the water component consists of or comprises water,
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water;
wherein the emulsion is obtained by an emulsification employing a low energy or ultra-low energy emulsification method;
(Be) an emulsion consisting of an ester component, a surfactant, a water component and one or more optional further components, wherein
the ester component is one or more compounds each characterized by the general Formula I: 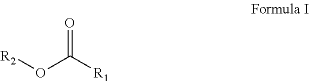
wherein R₁ represents a hydrocarbon group, which contains 30 or less carbon atoms and which is optionally substituted by 1 to 6 substituents each independently selected from fluorine, chlorine, and bromine,
R₂ represents an optionally substituted group selected from the group consisting of linear-chain C₁₋₁₂ alkyl groups, branched-chain C₃₋₁₂ alkyl groups, cyclic C₃₋₁₂ alkyl groups, linear-chain C₂₋₁₂ alkenyl groups, branched-chain C₃₋₁₂ alkenyl groups, cyclic C₅₋₁₂ alkenyl groups, linear-chain C₂₋₁₂ alkynyl groups, branched-chain C₄₋₁₂ alkynyl groups, C₅₋₁₄ aryl groups, 5-14-membered heteroaryl groups and 5-14 membered saturated or partly unsaturated non-aromatic heterocycles with 1 to 4 heteroatoms independently selected from N, O and S;
and wherein the ester component does not contain any iodine atoms as part of the R₁ group;
the surfactant is one or more non-ionic surfactant compounds;
the water component consists of or comprises water; and
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water;
(Ce1) an emulsion consisting of an ester component, a surfactant, a water component and one or more optional further components, wherein
the ester component is one or more compounds each characterized by the general Formula I: 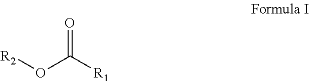
wherein R₁ is a linear C₁₄-C₂₀ alkyl group or a linear C₁₄-C₂₀ alkenyl group, each of which may optionally be substituted with 1, 2 or 3 halogen atoms selected from F, Cl, Br and I.
R₂ is selected from linear or branched C₁-C₆ alkyl groups or linear or branched C₁-C₆ alkenyl groups and preferably linear or branched C₁-C₄ alkyl groups or linear or branched C₁-C₄ alkenyl groups, each of which may be unsubstituted or carry 1, 2 or 3 substituents independently selected from F, Cl, Br, I, OH or —OCH₃;
the surfactant is one or more non-ionic surfactant compounds;
the water component consists of or comprises water; and
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water.
(Ce2) an emulsion consisting of an ester component, a surfactant, a water component and one or more optional further components, wherein
the ester component is one or more compounds each characterized by the general Formula I: 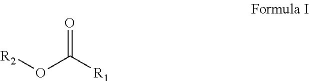
wherein R₁ represents a hydrocarbon group, which contains 30 or less carbon atoms and which is optionally substituted by 1 to 6 substituents each independently selected from fluorine, chlorine, bromine and iodine,
R₂ represents an optionally substituted group selected from the group consisting of linear-chain C₁₋₁₂ alkyl groups, branched-chain C₃₋₁₂ alkyl groups, cyclic C₃₋₁₂ alkyl groups, linear-chain C₂₋₁₂ alkenyl groups, branched-chain C₃₋₁₂ alkenyl groups, cyclic C₅₋₁₂ alkenyl groups, linear-chain C₂₋₁₂ alkynyl groups, branched-chain C₄₋₁₂ alkynyl groups, C₅₋₁₄ aryl groups, 5-14-membered heteroaryl groups and 5-14 membered saturated or partly unsaturated non-aromatic heterocycles with 1 to 4 heteroatoms independently selected from N, O and S;
the surfactant is one or more non-ionic surfactant compounds;
the water component is a water-based beverage other than purified water; and
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water;
(Ce3) an emulsion consisting of an ester component, a surfactant, a water component and one or more optional further components, wherein
the ester component is one or more compounds each characterized by the general Formula I: 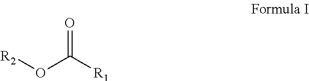
wherein R₁ represents a hydrocarbon group, which contains 30 or less carbon atoms and which is optionally substituted by 1 to 6 substituents each independently selected from fluorine, chlorine, bromine and iodine,
R₂ represents an optionally substituted group selected from the group consisting of linear-chain C₁₋₁₂ alkyl groups, branched-chain C₃₋₁₂ alkyl groups, cyclic C₃₋₁₂ alkyl groups, linear-chain C₂₋₁₂ alkenyl groups, branched-chain C₃₋₁₂ alkenyl groups, cyclic C₅₋₁₂ alkenyl groups, linear-chain C₂₋₁₂ alkynyl groups, branched-chain C₄₋₁₂ alkynyl groups, C₅₋₁₄ aryl groups, 5-14-membered heteroaryl groups and 5-14 membered saturated or partly unsaturated non-aromatic heterocycles with 1 to 4 heteroatoms independently selected from N, O and S;
the surfactant is one or more surfactant compounds each being selected from polyoxyethylene castor oils wherein the total number of oxyethylene units is preferably 4 or 5 or any higher number up to 20 or even up to 200, polyoxyethylene sorbitan fatty acid esters wherein the total number of oxyethylene units is preferably 4, 5 or any higher number up to 20, poloxamers such as poloxamer 124 and monoglycerides of fatty acids such as glyceryl monostearate;
the water component consists of or comprises water, such as a water-based beverage other than purified water; and
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water; and
(Ce4) an emulsion consisting of an ester component, a surfactant, a water component and one or more optional further components, wherein
the ester component is one or more compounds each characterized by the general Formula I: 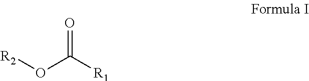
wherein R₁ represents a hydrocarbon group, which contains 30 or less carbon atoms and which is optionally substituted by 1 to 6 substituents each independently selected from fluorine, chlorine, bromine and iodine,
R₂ represents an optionally substituted group selected from the group consisting of linear-chain C₁₋₁₂ alkyl groups, branched-chain C₃₋₁₂ alkyl groups, cyclic C₃₋₁₂ alkyl groups, linear-chain C₂₋₁₂ alkenyl groups, branched-chain C₃₋₁₂ alkenyl groups, cyclic C₅₋₁₂ alkenyl groups, linear-chain C₂₋₁₂ alkynyl groups, branched-chain C₄₋₁₂ alkynyl groups, C₅₋₁₄ aryl groups, 5-14-membered heteroaryl groups and 5-14 membered saturated or partly unsaturated non-aromatic heterocycles with 1 to 4 heteroatoms independently selected from N, O and S;
the surfactant is one or more surfactant compounds each being selected from polyoxyethylene castor oils wherein the total number of oxyethylene units is preferably 4 or 5 or any higher number up to 20 or even up to 200, polyoxyethylene sorbitan fatty acid esters wherein the total number of oxyethylene units is preferably 4, 5 or any higher number up to 20;
the water component consists of or comprises water such as a water-based beverage other than purified water; and
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water.

2. A product for emulsification, which is selected from the following embodiments (Ape), (Bpe) and (Cpe),
(Ape) a product for emulsification, the product comprising a container containing a composition and instructions to emulsify by a low energy or ultra-low energy emulsification method such as manual shaking of the container, wherein the composition which is not in the form of an emulsion and which consists of an ester component, a surfactant, a water component and one or more optional further components, wherein
the ester component is one or more compounds each characterized by the general Formula I: 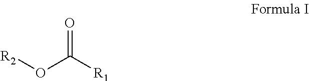
wherein R₁ represents a hydrocarbon group, which contains 30 or less carbon atoms and which is optionally substituted by 1 to 6 substituents each independently selected from fluorine, chlorine, bromine and iodine,
R₂ represents an optionally substituted group selected from the group consisting of linear-chain C₁₋₁₂ alkyl groups, branched-chain C₃₋₁₂ alkyl groups, cyclic C₃₋₁₂ alkyl groups, linear-chain C₂₋₁₂ alkenyl groups, branched-chain C₃₋₁₂ alkenyl groups, cyclic C₅₋₁₂ alkenyl groups, linear-chain C₂₋₁₂ alkynyl groups, branched-chain C₄₋₁₂ alkynyl groups, C₅₋₁₄ aryl groups, 5-14-membered heteroaryl groups and 5-14 membered saturated or partly unsaturated non-aromatic heterocycles with 1 to 4 heteroatoms independently selected from N, O and S;
the surfactant is one or more non-ionic surfactant compounds;
the water component consists of or comprises water,
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water;
(Bpe) a product for emulsification, the product comprising a container containing a composition and instructions to emulsify by a low energy or ultra-low energy emulsification method such as manual shaking of the container, wherein the composition which is not in the form of an emulsion and which consists of an ester component, a surfactant, a water component and one or more optional further components, wherein the ester component, a surfactant, a water component and one or more optional further components are as specified in claim 1, embodiment (Be),
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water;
(Cpe) a product for emulsification, the product comprising a container containing a composition and optionally instructions to emulsify by a low energy or ultra-low energy emulsification method such as manual shaking of the container, wherein the composition is not in the form of an emulsion and consists of an ester component, a surfactant, a water component and one or more optional further components, wherein the ester component, a surfactant, a water component and one or more optional further components are as specified in claim 1, embodiment (Ce1), (Ce2), (Ce3), or (Ce4),
wherein the relative amount of ester component is from more than 0 wt. % to 60 wt. % with respect to the total weight of ester component, surfactant and water, and
the relative amount of surfactant is from more than 0 wt. % to less than 40 wt. % with respect to the total weight of ester component, surfactant and water.

3. A premix product selected from the following embodiments (App), (Bpp) and (Cpp),
(App) a premix product for emulsification, the premix product comprising a container containing a premix composition and instructions to add a water component and emulsify by a low energy or ultra-low energy emulsification method such as manual shaking of the container, wherein the premix composition consists of an ester component, a surfactant and one or more optional further components, which are as specified in claim 1, embodiment (Ae), and the premix composition does not comprise a water component, wherein the weight ratio of ester component to surfactant is in the range of from 50:1 to 2:3, and/or wherein the weight of the surfactant relative to the total weight of ester component and surfactant is from 2 to 60%, or wherein the weight ratio of ester component to surfactant is in the range of from 10:1 to 2:3, and/or the weight of the surfactant relative to the total weight of ester component and surfactant is from 10 to 60%;
(Bpp) a premix product for emulsification, the premix product comprising a container containing a premix composition and instructions to add a water component and emulsify by a low energy or ultra-low energy emulsification method such as manual shaking of the container, wherein the premix composition consists of an ester component, a surfactant and one or more optional further components, which are as specified in claim 1, embodiment (Be), and the premix composition does not comprise a water component, wherein the weight ratio of ester component to surfactant is in the range of from 50:1 to 2:3, and/or wherein the weight of the surfactant relative to the total weight of ester component and surfactant is from 2 to 60%, or wherein the weight ratio of ester component to surfactant is in the range of from 10:1 to 2:3, and/or the weight of the surfactant relative to the total weight of ester component and surfactant is from 10 to 60%;
(Cpp) a premix product for emulsification, the premix product comprising a container containing a premix composition and instructions to add a water component and emulsify by a low energy or ultra-low energy emulsification method such as manual shaking of the container, wherein the premix composition consists of an ester component, a surfactant and one or more optional further components, which are as specified in claim 1, embodiment (Ce1), (Ce2), (Ce3), or (Ce4), and the premix composition does not comprise a water component, wherein the weight ratio of ester component to surfactant is in the range of from 50:1 to 2:3, and/or wherein the weight of the surfactant relative to the total weight of ester component and surfactant is from 2 to 60%, or wherein the weight ratio of ester component to surfactant is in the range of from 10:1 to 2:3, and/or the weight of the surfactant relative to the total weight of ester component and surfactant is from 10 to 60%.

4. A method for producing an emulsion which is selected from the following embodiments (Am), (Bm) and (Cm),
(Am) a method for producing an emulsion the method comprising the following steps:
(a) a step of providing a composition consisting of an ester component, a surfactant, a water component and one or more optional further components; and
(b) a step of emulsifying the composition of step (a) using a low or ultra-low energy emulsification method;
wherein the composition consisting of an ester component, a surfactant, a water component and one or more optional further components of step (a) is as specified in claim 2, embodiment (Ape);
(Bm) a method for producing the emulsion of claim 1, embodiment (Be), the method comprising the following steps:
(a) a step of providing a composition consisting of an ester component, a surfactant, a water component and one or more optional further components; and
(b) a step of emulsifying the composition of step (a) using a low or ultra-low energy emulsification method;
wherein the composition consisting of an ester component, a surfactant, a water component and one or more optional further components of step (a) is as specified in claim 2, embodiment (Bpe);
(Cm) a method for producing the emulsion of claim 1, embodiments (Ce1), (Ce2), (Ce3) or (Ce4), the method comprising the following steps:
(a) a step of providing a composition consisting of an ester component, a surfactant, a water component and one or more optional further components; and
(b) a step of emulsifying the composition of step (a) using a low or ultra-low energy emulsification method;
wherein the composition consisting of an ester component, a surfactant, a water component and one or more optional further components of step (a) is as specified in claim 2, embodiment (Cpe).

5. The emulsion of any of the embodiments of claim 1, product for emulsion of any of the embodiments of claim 2, premix of any of the embodiments of claim 3 or method of any of the embodiments of claim 4, wherein R₁ has 15 to 17 carbon atoms and is unsubstituted or is substituted by one, two, three, four, five or six substituents independently selected from fluorine, chlorine, and bromine.

6. The emulsion of claim 1, embodiments (Ae), (Be), (Ce1), (Ce2), (Ce3) or (Ce4), product for emulsion of claim 2, embodiments (Ape) or (Cpe), premix of claim 3, embodiments (App) or (Cpp) or method of claim 4, embodiments (Am) or (Cm), wherein R₁ has 15 to 17 carbon atoms and is unsubstituted or is substituted by one, two, three, four, five or six substituents independently selected from fluorine, chlorine, bromine and iodine.

7. The emulsion of any of the embodiments of claim 1 or of claim 5 or 6, product for emulsion of any of the embodiments of claim 2 or of claim 5 or 6, premix of any of the embodiments of claim 3 or of claim 5 or 6 or method of any of the embodiments of claim 4 or of claim 5 or 6, wherein R₂ is an unsubstituted group selected from a linear C₁₋₄ alkyl group, a branched C₃₋₄ alkyl group, a linear C₂₋₄ alkenyl group, a branched C₃₋₄ alkenyl group, a linear C₂₋₄ alkynyl group and a branched C₄ alkynyl group, preferably a C₁₋₄-alkyl group.

8. The emulsion of any of the embodiments of claim 1 or of claim 5, 6 or 7, product for emulsion of any of the embodiments of claim 2 or of claim 5, 6 or 7, premix of any of the embodiments of claim 3 or of claim 5, 6 or 7 or method of any of the embodiments of claim 4 or of claim 5, 6 or 7, wherein the surfactant is a single surfactant.

9. The emulsion of any of the embodiments of claim 1 or of claim 5, 6, 7 or 8, product for emulsion of any of the embodiments of claim 2 or of claim 5, 6, 7 or 8, premix of any of the embodiments of claim 3 or of claim 5, 6, 7 or 8 or method of any of the embodiments of claim 4 or of claim 5, 6, 7 or 8, wherein the surfactant is selected from compounds containing at least one hydrophilic group, independently selected from polyoxyethylene groups having from 3-60, preferably 4-40, ethyleneoxy units, monosaccharides, disaccharides, sugar alcohols, as well as at least one hydrophobic group independently selected from a saturated or unsaturated hydrocarbon group, preferably alkyl group, said hydrocarbon group having 8-20, preferably 12-18, carbon atoms.

10. The emulsion of any of the embodiments of claim 1 or of claim 5, 6, 7, 8 or 9, product for emulsion of any of the embodiments of claim 2 or of claim 5, 6, 7, 8 or 9 or method of any of the embodiments of claim 4 or of claim 5, 6, 7, 8 or 9, wherein the water component is selected from tap water, mineral water, carbonated water, soft drinks, preferably cola, lemonade or ginger ale, fruit juice, coffee, tea, milk or pharmaceutical grade aqueous solutions such as pharmaceutically acceptable buffers, preferably citrate, borate, phosphate, carbonate, acetate, saline, glucose, sucrose, mannitol and sorbitol solution.

11. The emulsion of any of the embodiments of claim 1 or of claim 5, 6, 7, 8, 9 or 10, product for emulsion of any of the embodiments of claim 2 or of claim 5, 6, 7, 8, 9 or 10, or method of any of the embodiments of claim 4 or of claim 5, 6, 7, 8, 9 or 10, wherein the total weight of ester component in relation to the total weight of ester component, surfactant and water in the emulsion of the invention is between 5 and 50%, preferably between 10 and 40%, the weight of surfactant in relation to the total weight of ester component, surfactant and water in the emulsion of the invention is more than 2% and 30% or less, preferably between 4 and 26%.

12. The emulsion of any of the embodiments of claim 1 or of claim 5, 6, 7, 8, 9, 10 or 11, product for emulsion of any of the embodiments of claim 2 or of claim 5, 6, 7, 8, 9, 10 or 11, or method of any of the embodiments of claim 4 or of claim 5, 6, 7, 8, 9, 10 or 11, wherein the ester component-to-surfactant ratio is between 40:1 and 40:60 (w/w), particularly between 90:10 and 40:60 (w/w), and the water-to-surfactant ratio is between 1000:1 and 55:45 (w/w) and the water-to-ester component ratio is between 1000:1 and 4:6 (w/w), preferably wherein the ester component-to-surfactant ratio is between 20:1 and 45:55 (w/w), particularly between 85:15 and 45:55 (w/w), and the water-to-surfactant ratio is between 400:1 and 65:35 and the water-to-ester component ratio is between 400:1 and 45:55 (w/w) and more preferably wherein the ester component-to-surfactant ratio is between 10:1 and 70:30 (w/w), particularly between 50:50 and 70:30 (w/w), and the water-to-surfactant ratio is between 100:1 and 80:20 (w/w) and the water-to-ester component ratio is between 100:1 and 1:1 (w/w).

13. The premix of any of the embodiments of claim 3 or of claim 5, 6, 7, 8, or 9, wherein the surfactant is present in a weight of 2 to 60%, preferably 4 to 55% and more preferably 9 to 50%, relative to the total weight of ester component and surfactant, or wherein the surfactant is present in a weight of 10 to 60%, preferably 15 to 55% and more preferably 30 to 50%, relative to the total weight of ester component and surfactant.

14. The method of any of the embodiments of claim 4 or of claim 5, 6, 7, 8, 9, 10, 11 or 12, wherein the emulsification of step (b) is performed using an ultra-low energy method and preferably by shaking by hand and/or wherein the duration of the emulsification of step (b) is 1.5 s to 120 s, preferably 1.5 to 60 s and more preferably 2 s to 30 s.

15. The emulsion of any of the embodiments of claim 1 or of claim 5, 6, 7, 8, 9, 10, 11 or 12, product for emulsion of any of the embodiments of claim 2 or of claim 5, 6, 7, 8, 9, 10, 11 or 12, premix of any of the embodiments of claim 3 or of claim 5, 6, 7, 8, 9 or 13 or method of any of the embodiments of claim 4 or of claim 5, 6, 7, 8, 9, 10, 11, 12 or 14, wherein at least one optional further component is present, which is soluble in the water component and/or wherein at least one optional further component is present, which is liquid and miscible with the ester component.
